SEQUENCE LISTING (37 sequences)

SEQ ID NO: 1 - protein, 629 aa, Homo sapiens [sequence omitted]

SEQ ID NO: 2 - protein, 629 aa, Homo sapiens
MPRYGASLRQSCPRSGREQGQDGTAGAPGLLWMGLALALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 3 - protein, 629 aa, Artificial sequence [sequence omitted]

SEQ ID NO: 4 - DNA, 1893 bp, Artificial Sequence
  Engineered hSMPD1.V36 coding sequence
atgcctagatacggcgcctctctgagacagagctgccctagatctggcagagagcagggccaagacggaacagctggtgctcctggactgctgtggatgggacttgctctggctttggctctggccctggctcttgctctgagcgattctagagtgctgtgggcccctgccgaagctcatcctttgtctccacaaggacaccccgccagactgcacagaatcgtgcccagactgagagatgtgttcggctggggcaacctgacctgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgacgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcaggtggctgtacgaagccatggccaaggcctgggaaccttggcttccagctgaagcactgcggaccctgagaatcggcggcttttatgccctgtctccttatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtcgacgagttcgaggtgttctacgacgaggaaaccctgagcagacctctggccgtggcatttctggccccaagcgccaccacatatatcggactgaaccccggctaccgggtgtaccagatcgacggcaattacagcggcagcagccacgtcgtgctggaccacgaaacctacatcctgaatctgacccaggccaacattcccggcgctatccctcattggcagctgctgtacagagccagagagacatacggcctgcctaacacactgccaaccgcctggcacaacctggtgtacagaatgagaggcgacatgcagctgtttcagaccttctggttcctgtaccacaaggggcaccctccaagcgagccttgtggcacaccttgtagactggccactctgtgcgctcagctgtccgccagagctgattctcctgctctgtgcagacacctgatgcctgatggaagcctgcctgaggctcagagcctttggcctagacctctgttctgctgatga

SEQ ID NO: 5 - DNA, 1893 bp, Artificial sequence
  hSMPD1.V36
atgcctagatacggcgcctctctgagacagagctgccctagatctggcagagagcagggccaagacggaacagctggtgctcctggactgctgtggatgggacttgtcctggctttggctctggccctggctcttgctctgagcgattctagagtgctgtgggcccctgccgaagctcatcctttgtctccacaaggacaccccgccagactgcacagaatcgtgcccagactgagagatgtgttcggctggggcaacctgacctgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgacgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcaggtggctgtacgaagccatggccaaggcctgggaaccttggcttccagctgaagcactgcggaccctgagaatcggcggcttttatgccctgtctccttatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtcgacgagttcgaggtgttctacgacgaggaaaccctgagcagacctctggccgtggcatttctggccccaagcgccaccacatatatcggactgaaccccggctaccgggtgtaccagatcgacggcaattacagcggcagcagccacgtcgtgctggaccacgaaacctacatcctgaatctgacccaggccaacattcccggcgctatccctcattggcagctgctgtacagagccagagagacatacggcctgcctaacacactgccaaccgcctggcacaacctggtgtacagaatgagaggcgacatgcagctgtttcagaccttctggttcctgtaccacaaggggcaccctccaagcgagccttgtggcacaccttgtagactggccactctgtgcgctcagctgtccgccagagctgattctcctgctctgtgcagacacctgatgcctgatggaagcctgcctgaggctcagagcctttggcctagacctctgttctgctgatga

SEQ ID NO: 6 - DNA, 282 bp, Artificial Sequence
  CB promoter
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 7 - DNA, 127 bp, Artificial sequence
  Rabbit globin polyA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 8 - DNA, 3801 bp, Artificial Sequence
  CB7.CI.hSMPD1co(TY).RBG
  misc_feature (1)..(382): CMV IE enhancer
  misc_feature (1)..(666): CB7 hybrid promoter
  misc_feature (385)..(666): CB promoter
  misc_feature (759)..(1731): chicken beta actin intron
  misc_feature (1749)..(3641): hSMPD1.V36A.co
  misc_feature (3675)..(3801): Rabbit beta globin polyA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgccaccatgcctagatacggcgcctctctgagacagagctgccctagatctggcagagagcagggccaagacggaacagctggtgctcctggactgctgtggatgggacttgctctggctttggctctggccctggctcttgctctgagcgattctagagtgctgtgggcccctgccgaagctcatcctttgtctccacaaggacaccccgccagactgcacagaatcgtgcccagactgagagatgtgttcggctggggcaacctgacctgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgacgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcaggtggctgtacgaagccatggccaaggcctgggaaccttggcttccagctgaagcactgcggaccctgagaatcggcggcttttatgccctgtctccttatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtcgacgagttcgaggtgttctacgacgaggaaaccctgagcagacctctggccgtggcatttctggccccaagcgccaccacatatatcggactgaaccccggctaccgggtgtaccagatcgacggcaattacagcggcagcagccacgtcgtgctggaccacgaaacctacatcctgaatctgacccaggccaacattcccggcgctatccctcattggcagctgctgtacagagccagagagacatacggcctgcctaacacactgccaaccgcctggcacaacctggtgtacagaatgagaggcgacatgcagctgtttcagaccttctggttcctgtaccacaaggggcaccctccaagcgagccttgtggcacaccttgtagactggccactctgtgcgctcagctgtccgccagagctgattctcctgctctgtgcagacacctgatgcctgatggaagcctgcctgaggctcagagcctttggcctagacctctgttctgctgatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 9 - DNA, 4216 bp, Artificial sequence
  ITR.CB7.CI.hSMPD1co(TY).RBG.ITR
  misc_feature (1)..(130): 5′ ITR
  misc_feature (198)..(579): CMV IE promoter
  misc_feature (198)..(863): CB7 hybrid promoter
  misc_feature (582)..(863): CB promoter
  misc_feature (956)..(1928): chicken beta-actin intron
  misc_feature (1946)..(3838): hSMPD1.V36A.co
  misc_feature (3872)..(3998): rabbit gobin polyA
  misc_feature (4087)..(4216): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgccaccatgcctagatacggcgcctctctgagacagagctgccctagatctggcagagagcagggccaagacggaacagctggtgctcctggactgctgtggatgggacttgctctggctttggctctggccctggctcttgctctgagcgattctagagtgctgtgggcccctgccgaagctcatcctttgtctccacaaggacaccccgccagactgcacagaatcgtgcccagactgagagatgtgttcggctggggcaacctgacctgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgacgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcaggtggctgtacgaagccatggccaaggcctgggaaccttggcttccagctgaagcactgcggaccctgagaatcggcggcttttatgccctgtctccttatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtcgacgagttcgaggtgttctacgacgaggaaaccctgagcagacctctggccgtggcatttctggccccaagcgccaccacatatatcggactgaaccccggctaccgggtgtaccagatcgacggcaattacagcggcagcagccacgtcgtgctggaccacgaaacctacatcctgaatctgacccaggccaacattcccggcgctatccctcattggcagctgctgtacagagccagagagacatacggcctgcctaacacactgccaaccgcctggcacaacctggtgtacagaatgagaggcgacatgcagctgtttcagaccttctggttcctgtaccacaaggggcaccctccaagcgagccttgtggcacaccttgtagactggccactctgtgcgctcagctgtccgccagagctgattctcctgctctgtgcagacacctgatgcctgatggaagcctgcctgaggctcagagcctttggcctagacctctgttctgctgatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 10 - DNA, 3801 bp, Artificial sequence
  CB7.CI.hSMPD1.V36co.RBG
  misc_feature (1)..(382): CMV IE enhancer
  misc_feature (1)..(666): CB7 hybrid promoter
  misc_feature (385)..(666): CB promoter
  misc_feature (759)..(1731): chicken beta actin inron
  misc_feature (1749)..(3641): hSMPD1.V36.co
  misc_feature (3675)..(3801): rabbit beta globin polyA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgccaccatgcctagatacggcgcctctctgagacagagctgccctagatctggcagagagcagggccaagacggaacagctggtgctcctggactgctgtggatgggacttgtcctggctttggctctggccctggctcttgctctgagcgattctagagtgctgtgggcccctgccgaagctcatcctttgtctccacaaggacaccccgccagactgcacagaatcgtgcccagactgagagatgtgttcggctggggcaacctgacctgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgacgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcaggtggctgtacgaagccatggccaaggcctgggaaccttggcttccagctgaagcactgcggaccctgagaatcggcggcttttatgccctgtctccttatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtcgacgagttcgaggtgttctacgacgaggaaaccctgagcagacctctggccgtggcatttctggccccaagcgccaccacatatatcggactgaaccccggctaccgggtgtaccagatcgacggcaattacagcggcagcagccacgtcgtgctggaccacgaaacctacatcctgaatctgacccaggccaacattcccggcgctatccctcattggcagctgctgtacagagccagagagacatacggcctgcctaacacactgccaaccgcctggcacaacctggtgtacagaatgagaggcgacatgcagctgtttcagaccttctggttcctgtaccacaaggggcaccctccaagcgagccttgtggcacaccttgtagactggccactctgtgcgctcagctgtccgccagagctgattctcctgctctgtgcagacacctgatgcctgatggaagcctgcctgaggctcagagcctttggcctagacctctgttctgctgatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 11 - DNA, 4216 bp, Artificial sequence
  ITR.CB7.CI.hSMPD1coV36.RBG.ITR
  misc_feature (1)..(130): 5′ ITR
  misc_feature (198)..(863): CB7 hybrid promoter
  misc_feature (198)..(579): CMV IE promoter
  misc_feature (583)..(863): CB promoter
  misc_feature (956)..(1928): chicken beta-actin intron
  misc_feature (1946)..(3838): hSMPD1,V36co
  misc_feature (3872)..(3998): rabbit globin poly A
  misc_feature (4088)..(4216): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgccaccatgcctagatacggcgcctctctgagacagagctgccctagatctggcagagagcagggccaagacggaacagctggtgctcctggactgctgtggatgggacttgtcctggctttggctctggccctggctcttgctctgagcgattctagagtgctgtgggcccctgccgaagctcatcctttgtctccacaaggacaccccgccagactgcacagaatcgtgcccagactgagagatgtgttcggctggggcaacctgacctgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgacgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcaggtggctgtacgaagccatggccaaggcctgggaaccttggcttccagctgaagcactgcggaccctgagaatcggcggcttttatgccctgtctccttatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtcgacgagttcgaggtgttctacgacgaggaaaccctgagcagacctctggccgtggcatttctggccccaagcgccaccacatatatcggactgaaccccggctaccgggtgtaccagatcgacggcaattacagcggcagcagccacgtcgtgctggaccacgaaacctacatcctgaatctgacccaggccaacattcccggcgctatccctcattggcagctgctgtacagagccagagagacatacggcctgcctaacacactgccaaccgcctggcacaacctggtgtacagaatgagaggcgacatgcagctgtttcagaccttctggttcctgtaccacaaggggcaccctccaagcgagccttgtggcacaccttgtagactggccactctgtgcgctcagctgtccgccagagctgattctcctgctctgtgcagacacctgatgcctgatggaagcctgcctgaggctcagagcctttggcctagacctctgttctgctgatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 12 - DNA, 1229 bp, Artificial sequence
  UbC Promoter
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggta

SEQ ID NO: 13 - DNA, 232 bp, Artificial sequence
  SV40 late polyA signal
cgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcg

SEQ ID NO: 14 - DNA, 3695 bp, Artificial sequence
  UbC.hSMPD1co.SV40
  misc_feature (1)..(1229): UbC promoter
  misc_feature (1323)..(1455): Promega Chimeric Intron
  misc_feature (1540)..(3432): hSMPD1.V36A.co
  misc_feature (3464)..(3695): SV40 late polyA signal
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagaattcacgcgtgccaccatgcctagatacggcgcctctctgagacagagctgccctagatctggcagagagcagggccaagacggaacagctggtgctcctggactgctgtggatgggacttgctctggctttggctctggccctggctcttgctctgagcgattctagagtgctgtgggcccctgccgaagctcatcctttgtctccacaaggacaccccgccagactgcacagaatcgtgcccagactgagagatgtgttcggctggggcaacctgacctgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgacgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcaggtggctgtacgaagccatggccaaggcctgggaaccttggcttccagctgaagcactgcggaccctgagaatcggcggcttttatgccctgtctccttatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtcgacgagttcgaggtgttctacgacgaggaaaccctgagcagacctctggccgtggcatttctggccccaagcgccaccacatatatcggactgaaccccggctaccgggtgtaccagatcgacggcaattacagcggcagcagccacgtcgtgctggaccacgaaacctacatcctgaatctgacccaggccaacattcccggcgctatccctcattggcagctgctgtacagagccagagagacatacggcctgcctaacacactgccaaccgcctggcacaacctggtgtacagaatgagaggcgacatgcagctgtttcagaccttctggttcctgtaccacaaggggcaccctccaagcgagccttgtggcacaccttgtagactggccactctgtgcgctcagctgtccgccagagctgattctcctgctctgtgcagacacctgatgcctgatggaagcctgcctgaggctcagagcctttggcctagacctctgttctgctgatgactcgagcacgtgtgatgactcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcg

SEQ ID NO: 15 - DNA, 4079 bp, Artificial sequence
  ITR.UbC.hSMPD1co.SV40.ITR
  misc_feature (1)..(130): 5′ ITR
  misc_feature (191)..(1419): UbC promoter
  misc_feature (1513)..(1645): Promega chimeric intron
  misc_feature (1730)..(3622): hSMPD1.V36Aco
  misc_feature (3654)..(3885): SV40 late polyA signal
  misc_feature (3950)..(4079): 3 ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagaattcacgcgtgccaccatgcctagatacggcgcctctctgagacagagctgccctagatctggcagagagcagggccaagacggaacagctggtgctcctggactgctgtggatgggacttgctctggctttggctctggccctggctcttgctctgagcgattctagagtgctgtgggcccctgccgaagctcatcctttgtctccacaaggacaccccgccagactgcacagaatcgtgcccagactgagagatgtgttcggctggggcaacctgacctgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgacgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcaggtggctgtacgaagccatggccaaggcctgggaaccttggcttccagctgaagcactgcggaccctgagaatcggcggcttttatgccctgtctccttatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtcgacgagttcgaggtgttctacgacgaggaaaccctgagcagacctctggccgtggcatttctggccccaagcgccaccacatatatcggactgaaccccggctaccgggtgtaccagatcgacggcaattacagcggcagcagccacgtcgtgctggaccacgaaacctacatcctgaatctgacccaggccaacattcccggcgctatccctcattggcagctgctgtacagagccagagagacatacggcctgcctaacacactgccaaccgcctggcacaacctggtgtacagaatgagaggcgacatgcagctgtttcagaccttctggttcctgtaccacaaggggcaccctccaagcgagccttgtggcacaccttgtagactggccactctgtgcgctcagctgtccgccagagctgattctcctgctctgtgcagacacctgatgcctgatggaagcctgcctgaggctcagagcctttggcctagacctctgttctgctgatgactcgagcacgtgtgatgactcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - DNA, 2211 bp, Artificial Sequence
  AAVhu68 vp1 capsid of Homo Sapiens origin
  CDS (1)..(2211)
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 17 - protein, 736 aa, Artificial Sequence
  Synthetic Construct
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 18 - DNA, 4312 bp, Artificial Sequence
  ITR.CB7.CI.hSMPD1coV2.4xmiR182.rBG.ITR
  misc_feature (1)..(130): 5′ ITR
  misc_feature (198)..(579): CMV IE enhancer
  misc_feature (198)..(863): CB7 hybrid promoter
  misc_feature (582)..(863): CB promoter
  misc_feature (956)..(1928): chicken beta actin intron
  misc_feature (1934)..(3843): hSMPD1coV2
  misc_feature (3845)..(3868): miR182
  misc_feature (3873)..(3896): miR182
  misc_feature (3903)..(3926): miR182
  misc_feature (3933)..(3956): miR182
  misc_feature (3968)..(4094): Rabbit beta globin polyA
  misc_feature (4183)..(4312): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgcctagatacggcgcctctctgagacagagctgccctagatctggcagagagcagggccaagacggaacagctggtgctcctggactgctgtggatgggacttgctctggctttggctctggccctggctcttgctctgagcgattctagagtgctgtgggcccctgccgaagctcatcctttgtctccacaaggacaccccgccagactgcacagaatcgtgcccagactgagagatgtgttcggctggggcaacctgacctgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgacgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcaggtggctgtacgaagccatggccaaggcctgggaaccttggcttccagctgaagcactgcggaccctgagaatcggcggcttttatgccctgtctccttatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtcgacgagttcgaggtgttctacgacgaggaaaccctgagcagacctctggccgtggcatttctggccccaagcgccaccacatatatcggactgaaccccggctaccgggtgtaccagatcgacggcaattacagcggcagcagccacgtcgtgctggaccacgaaacctacatcctgaatctgacccaggccaacattcccggcgctatccctcattggcagctgctgtacagagccagagagacatacggcctgcctaacacactgccaaccgcctggcacaacctggtgtacagaatgagaggcgacatgcagctgtttcagaccttctggttcctgtaccacaaggggcaccctccaagcgagccttgtggcacaccttgtagactggccactctgtgcgctcagctgtccgccagagctgattctcctgctctgtgcagacacctgatgcctgacggaagcctgcctgaggctcagagcctttggcctagacctctgttctgctgataaggtaccagtgtgagttctaccattgccaaaggatagtgtgagttctaccattgccaaacacgtgagtgtgagttctaccattgccaaagcatgcagtgtgagttctaccattgccaaagcggccgatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 19 - DNA, 3897 bp, Artificial Sequence
  CB7.CI.hSMPD1coV2.4xmiR182.rBG
  misc_feature (1)..(382): CMV IE enhancer
  misc_feature (1)..(666): CB7 hybrid promoter
  misc_feature (385)..(666): CB promoter
  misc_feature (759)..(1731): chicken beta actin intron
  misc_feature (1743)..(1748): Kozak
  misc_feature (1749)..(3641): hSMPD1coV2
  misc_feature (3648)..(3671): miR182
  misc_feature (3676)..(3699): miR182
  misc_feature (3706)..(3729): miR182
  misc_feature (3736)..(3759): miR182
  misc_feature (3771)..(3897): rabbit globin polyA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgcctagatacggcgcctctctgagacagagctgccctagatctggcagagagcagggccaagacggaacagctggtgctcctggactgctgtggatgggacttgctctggctttggctctggccctggctcttgctctgagcgattctagagtgctgtgggcccctgccgaagctcatcctttgtctccacaaggacaccccgccagactgcacagaatcgtgcccagactgagagatgtgttcggctggggcaacctgacctgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgacgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcaggtggctgtacgaagccatggccaaggcctgggaaccttggcttccagctgaagcactgcggaccctgagaatcggcggcttttatgccctgtctccttatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtcgacgagttcgaggtgttctacgacgaggaaaccctgagcagacctctggccgtggcatttctggccccaagcgccaccacatatatcggactgaaccccggctaccgggtgtaccagatcgacggcaattacagcggcagcagccacgtcgtgctggaccacgaaacctacatcctgaatctgacccaggccaacattcccggcgctatccctcattggcagctgctgtacagagccagagagacatacggcctgcctaacacactgccaaccgcctggcacaacctggtgtacagaatgagaggcgacatgcagctgtttcagaccttctggttcctgtaccacaaggggcaccctccaagcgagccttgtggcacaccttgtagactggccactctgtgcgctcagctgtccgccagagctgattctcctgctctgtgcagacacctgatgcctgacggaagcctgcctgaggctcagagcctttggcctagacctctgttctgctgataaggtaccagtgtgagttctaccattgccaaaggatagtgtgagttctaccattgccaaacacgtgagtgtgagttctaccattgccaaagcatgcagtgtgagttctaccattgccaaagcggccgatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 20 - DNA, 4243 bp, Artificial Sequence
  ITR.CB7.CI.hSMPD1cov2.RBG.ITR
  misc_feature (1)..(130): 5′ITR
  misc_feature (198)..(579): CMV IE enhancer
  misc_feature (198)..(863): CB7 hybrid promoter
  misc_feature (582)..(863): CB promoter
  misc_feature (956)..(1928): chicken beta actin intron
  misc_feature (1940)..(19456): Kozak
  misc_feature (1946)..(3838): hSMPD1coV2
  misc_feature (3899)..(4025): rabbit beta globin polyA
  misc_feature (4114)..(4243): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgcctagatacggcgcctctctgagacagagctgccctagatctggcagagagcagggccaagacggaacagctggtgctcctggactgctgtggatgggacttgctctggctttggctctggccctggctcttgctctgagcgattctagagtgctgtgggcccctgccgaagctcatcctttgtctccacaaggacaccccgccagactgcacagaatcgtgcccagactgagagatgtgttcggctggggcaacctgacctgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgacgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcaggtggctgtacgaagccatggccaaggcctgggaaccttggcttccagctgaagcactgcggaccctgagaatcggcggcttttatgccctgtctccttatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtcgacgagttcgaggtgttctacgacgaggaaaccctgagcagacctctggccgtggcatttctggccccaagcgccaccacatatatcggactgaaccccggctaccgggtgtaccagatcgacggcaattacagcggcagcagccacgtcgtgctggaccacgaaacctacatcctgaatctgacccaggccaacattcccggcgctatccctcattggcagctgctgtacagagccagagagacatacggcctgcctaacacactgccaaccgcctggcacaacctggtgtacagaatgagaggcgacatgcagctgtttcagaccttctggttcctgtaccacaaggggcaccctccaagcgagccttgtggcacaccttgtagactggccactctgtgcgctcagctgtccgccagagctgattctcctgctctgtgcagacacctgatgcctgacggaagcctgcctgaggctcagagcctttggcctagacctctgttctgctgataaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 21 - DNA, 3828 bp, Artificial Sequence
  CB7.CI.hSMPD1cov2.RBG
  misc_feature (1)..(382): CMV IE enhancer
  misc_feature (1)..(666): CB7 hybrid promoter
  misc_feature (385)..(666): CB promoter
  misc_feature (759)..(1731): chicken beta-actin intron
  misc_feature (1743)..(1748): Kozak
  misc_feature (1749)..(3641): hSMPD1coV2
  misc_feature (3702)..(3828): Rabbit beta globin polyA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgcctagatacggcgcctctctgagacagagctgccctagatctggcagagagcagggccaagacggaacagctggtgctcctggactgctgtggatgggacttgctctggctttggctctggccctggctcttgctctgagcgattctagagtgctgtgggcccctgccgaagctcatcctttgtctccacaaggacaccccgccagactgcacagaatcgtgcccagactgagagatgtgttcggctggggcaacctgacctgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgacgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcaggtggctgtacgaagccatggccaaggcctgggaaccttggcttccagctgaagcactgcggaccctgagaatcggcggcttttatgccctgtctccttatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtcgacgagttcgaggtgttctacgacgaggaaaccctgagcagacctctggccgtggcatttctggccccaagcgccaccacatatatcggactgaaccccggctaccgggtgtaccagatcgacggcaattacagcggcagcagccacgtcgtgctggaccacgaaacctacatcctgaatctgacccaggccaacattcccggcgctatccctcattggcagctgctgtacagagccagagagacatacggcctgcctaacacactgccaaccgcctggcacaacctggtgtacagaatgagaggcgacatgcagctgtttcagaccttctggttcctgtaccacaaggggcaccctccaagcgagccttgtggcacaccttgtagactggccactctgtgcgctcagctgtccgccagagctgattctcctgctctgtgcagacacctgatgcctgacggaagcctgcctgaggctcagagcctttggcctagacctctgttctgctgataaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 22 - DNA, 1893 bp, Artificial Sequence
  hSMPD1coV2
  CDS (1)..(1893): hSMPD1coV2
atgcctagatacggcgcctctctgagacagagctgccctagatctggcagagagcagggccaagacggaacagctggtgctcctggactgctgtggatgggacttgctctggctttggctctggccctggctcttgctctgagcgattctagagtgctgtgggcccctgccgaagctcatcctttgtctccacaaggacaccccgccagactgcacagaatcgtgcccagactgagagatgtgttcggctggggcaacctgacctgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgacgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcaggtggctgtacgaagccatggccaaggcctgggaaccttggcttccagctgaagcactgcggaccctgagaatcggcggcttttatgccctgtctccttatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtcgacgagttcgaggtgttctacgacgaggaaaccctgagcagacctctggccgtggcatttctggccccaagcgccaccacatatatcggactgaaccccggctaccgggtgtaccagatcgacggcaattacagcggcagcagccacgtcgtgctggaccacgaaacctacatcctgaatctgacccaggccaacattcccggcgctatccctcattggcagctgctgtacagagccagagagacatacggcctgcctaacacactgccaaccgcctggcacaacctggtgtacagaatgagaggcgacatgcagctgtttcagaccttctggttcctgtaccacaaggggcaccctccaagcgagccttgtggcacaccttgtagactggccactctgtgcgctcagctgtccgccagagctgattctcctgctctgtgcagacacctgatgcctgacggaagcctgcctgaggctcagagcctttggcctagacctctgttctgctgataa

SEQ ID NO: 23 - protein, 629 aa, Artificial Sequence; identical to SEQ ID NO: 2
  Synthetic Construct

SEQ ID NO: 24 - DNA, 24 bp, Artificial Sequence
  miR182
agtgtgagttctaccattgccaaa

SEQ ID NO: 25 - DNA, 382 bp, Artificial Sequence
  cytomegalovirus (CMV) IE enhancer
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 26 - DNA, 666 bp, Artificial Sequence
  CB7 hybrid promoter
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 27 - DNA, 973 bp, Artificial Sequence
  chicken beta-actin intron
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 28 - DNA, 112 bp, Artificial Sequence
  4 tandem repeats of miR182 with linker sequences
  misc_feature (1)..(24): miR182
  misc_feature (29)..(52): miR182
  misc_feature (59)..(82): miR182
  misc_feature (89)..(112): miR182
agtgtgagttctaccattgccaaaggatagtgtgagttctaccattgccaaacacgtgagtgtgagttctaccattgccaaagcatgcagtgtgagttctaccattgccaaa

SEQ ID NO: 29 - DNA, 2211 bp, adeno-associated virus hu68
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgacggcagtcaggcggtgggccgctcatccttctactgcctggagtacttcccttcgcagatgctgaggactggcaacaacttccagttcagctacgagttcgagaacgtccctttccacagcagctacgcccacagccagagtttggaccgcttgatgaaccctctgatcgaccagtacctgtactacctgtcaaagacgatcaacggttctggccagaaccagcagacgctgaagttcagcgtggccgggcctagcaacatggccgtccagggcagaaactacatccctgggcccagctaccggcagcagagagtctcaaccactgtgactcagaacaacaacagtgagttcgcctggcctggcgccagctcttgggccctcaacggccgcaactcgctgatgaacccaggcccagccatggccagtcacaaggagggcgaggaccgtttcttccctttgtctggctctctgatcttcggcaagcaggggaccggcagagacaacgtggacgcggacaaggtcatgatcacgaacgaggaggagatcaagaccaccaaccctgtggcaaccgagtcctacggccaggtggcaaccaaccaccagagcgcccaggcacaggcgcagactggctgggtccagaaccaggggatcctgcctggcatggtgtggcaggaccgtgacgtgtacctgcagggccctatctgggcaaagatccctcacacggacggcaacttccacccttctcctctgatgggcggcttcggcatgaagcacccgcctcctcagatcctcatcaagaacactccggtcccggcagaccctccgacggccttcaacaaggacaagctgaactcattcatcactcagtactccactggccaggtcagcgtggagatcgagtgggagctgcagaaggagaacagcaagcgttggaacccagagatccagtacacttccaactactacaagtctaacaacgtggagttcgccgtcaacactgagggtgtgtacagtgagcctcgccctatcggcacccggtacctcacccgaaacttgtga

SEQ ID NO: 30 - protein, 46 aa, Artificial Sequence
  native signal peptide of hSMPD1 (aa 1 to 46)
MPRYGASLRQSCPRSGREQGQDGTAGAPGLLWMGLALALALALALA

SEQ ID NO: 31 - protein, 583 aa, Artificial Sequence
  hSMPD1 mature protein (aa 47 to about 629)
LSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 32 - protein, 599 aa, adeno-associated virus hu68 VP2
TAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - DNA, 1800 bp, adeno-associated virus hu68 VP2
acggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 34 - DNA, 1800 bp, adeno-associated virus hu68 VP2
acggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgacggcagtcaggcggtgggccgctcatccttctactgcctggagtacttcccttcgcagatgctgaggactggcaacaacttccagttcagctacgagttcgagaacgtccctttccacagcagctacgcccacagccagagtttggaccgcttgatgaaccctctgatcgaccagtacctgtactacctgtcaaagacgatcaacggttctggccagaaccagcagacgctgaagttcagcgtggccgggcctagcaacatggccgtccagggcagaaactacatccctgggcccagctaccggcagcagagagtctcaaccactgtgactcagaacaacaacagtgagttcgcctggcctggcgccagctcttgggccctcaacggccgcaactcgctgatgaacccaggcccagccatggccagtcacaaggagggcgaggaccgtttcttccctttgtctggctctctgatcttcggcaagcaggggaccggcagagacaacgtggacgcggacaaggtcatgatcacgaacgaggaggagatcaagaccaccaaccctgtggcaaccgagtcctacggccaggtggcaaccaaccaccagagcgcccaggcacaggcgcagactggctgggtccagaaccaggggatcctgcctggcatggtgtggcaggaccgtgacgtgtacctgcagggccctatctgggcaaagatccctcacacggacggcaacttccacccttctcctctgatgggcggcttcggcatgaagcacccgcctcctcagatcctcatcaagaacactccggtcccggcagaccctccgacggccttcaacaaggacaagctgaactcattcatcactcagtactccactggccaggtcagcgtggagatcgagtgggagctgcagaaggagaacagcaagcgttggaacccagagatccagtacacttccaactactacaagtctaacaacgtggagttcgccgtcaacactgagggtgtgtacagtgagcctcgccctatcggcacccggtacctcacccgaaacttgtga

SEQ ID NO: 35 - protein, 534 aa, adeno-associated virus hu68 VP3
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 36 - DNA, 1605 bp, adeno-associated virus hu68 VP3
atggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 37 - DNA, 1605 bp, adeno-associated virus hu68 VP3
atggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgacggcagtcaggcggtgggccgctcatccttctactgcctggagtacttcccttcgcagatgctgaggactggcaacaacttccagttcagctacgagttcgagaacgtccctttccacagcagctacgcccacagccagagtttggaccgcttgatgaaccctctgatcgaccagtacctgtactacctgtcaaagacgatcaacggttctggccagaaccagcagacgctgaagttcagcgtggccgggcctagcaacatggccgtccagggcagaaactacatccctgggcccagctaccggcagcagagagtctcaaccactgtgactcagaacaacaacagtgagttcgcctggcctggcgccagctcttgggccctcaacggccgcaactcgctgatgaacccaggcccagccatggccagtcacaaggagggcgaggaccgtttcttccctttgtctggctctctgatcttcggcaagcaggggaccggcagagacaacgtggacgcggacaaggtcatgatcacgaacgaggaggagatcaagaccaccaaccctgtggcaaccgagtcctacggccaggtggcaaccaaccaccagagcgcccaggcacaggcgcagactggctgggtccagaaccaggggatcctgcctggcatggtgtggcaggaccgtgacgtgtacctgcagggccctatctgggcaaagatccctcacacggacggcaacttccacccttctcctctgatgggcggcttcggcatgaagcacccgcctcctcagatcctcatcaagaacactccggtcccggcagaccctccgacggccttcaacaaggacaagctgaactcattcatcactcagtactccactggccaggtcagcgtggagatcgagtgggagctgcagaaggagaacagcaagcgttggaacccagagatccagtacacttccaactactacaagtctaacaacgtggagttcgccgtcaacactgagggtgtgtacagtgagcctcgccctatcggcacccggtacctcacccgaaacttgtga

(19) United States
(12) Patent Application Publication
(10) Pub. No.: US 2024/0115733 A1
(43) Pub. Date: Apr. 11, 2024

(54) COMPOSITIONS AND METHODS FOR TREATMENT OF NIEMANN PICK TYPE A DISEASE

(71) Applicant: The Trustees of the University of Pennsylvania, Philadelphia, PA (US)

(72) Inventors: James M. WILSON, Philadelphia, PA (US); Juliette HORDEAUX, Philadelphia, PA (US); Ting YU, Changzhou (CN)

(21) Appl. No.: 18/263,085

(22) PCT Filed: Feb. 1, 2022
(86) PCT No.: PCT/US2022/014742

§ 371 Date: Jul. 26, 2023

Related U.S. Application Data
(60) Provisional application No. 63/144,103, filed on Feb. 1, 2021.
(60) Provisional application No. 63/286,939, filed on Dec. 7, 2021.

(51) Int. Cl. A61K 48/00; A61K 9/00; A61P 3/06; C12N 9/16; C12N 15/86

(52) U.S. Cl. CPC A61K 48/005; A61K 9/0019; A61K 9/0085; A61K 48/0075; A61P 3/06; C12N 9/16; C12N 15/86; C12N 2750/14143; C12Y 301/04012

(57) ABSTRACT

Provided herein are polynucleotide sequences encoding human acid sphingomyelinase (SMPD1) and expression cassettes containing these coding sequences. Also provided are vectors, such as recombinant adeno-associated virus (rAAV) vectors having vector genomes that include an engineered SMPD1 coding sequence operably linked to one or more regulatory sequences. Further, compositions containing these expression cassettes and rAAV are provided, as well as methods for the use of these compositions for treatment of Niemann Pick Type A disease.

### BACKGROUND OF THE INVENTION

Niemann-Pick disease is an inherited condition involving lipid metabolism, which is the breakdown, transport, and use of fats and cholesterol in the body. In people with this condition, abnormal lipid metabolism causes harmful amounts of lipids to accumulate in the spleen, liver, lungs, bone marrow, and brain. Niemann-Pick disease type A and B are caused by different sets of mutations in the SMPD1 gene. This gene encodes an enzyme called acid sphingomyelinase. This enzyme is found in lysosomes, which are compartments within cells that break down and recycle different types of molecules. Acid sphingomyelinase is responsible for the conversion of a fat (lipid) called sphingomyelin into another type of lipid called ceramide. Mutations in SMPD1 lead to a shortage of acid sphingomyelinase, which results in reduced break down of sphingomyelin, causing this fat to accumulate in cells. This fat buildup causes cells to malfunction and eventually die. Over time, cell loss impairs function of tissues and organs including the brain, lungs, spleen, and liver in people with Niemann-Pick disease types A and B.

Niemann-Pick disease type A appears during infancy and is characterized by an enlarged liver and spleen (hepatosplenomegaly), failure to gain weight and grow at the expected rate (failure to thrive), and progressive deterioration of the nervous system. Due to the involvement of the nervous system, Niemann-Pick disease type A is also known as the neurological type. Infants with Niemann-Pick disease type A usually develop an enlarged liver and spleen (hepatosplenomegaly) by age 3 months and fail to gain weight and grow at the expected rate (failure to thrive). The affected children develop normally until around age 1 year when they experience a progressive loss of mental abilities and movement (psychomotor regression). Children with Niemann-Pick disease type A also develop widespread lung damage (interstitial lung disease) that can cause recurrent lung infections and eventually lead to respiratory failure. All affected children have an eye abnormality called a cherry-red spot, which can be identified with an eye examination. Children with Niemann-Pick disease type A (NPA) generally do not survive past early childhood. There is currently no effective treatment for this condition.

A need in the art exists for compositions and methods for safe and effective treatment of patients with NPA disease.

### SUMMARY OF THE INVENTION

In one aspect, provided is a recombinant AAV (rAAV) comprising an AAVhu68 capsid having packaged therein a vector genome, wherein the vector genome comprises a human sphingomyelin phosphodiesterase 1 (hSMPD1) coding sequence and regulatory sequences which direct expression of a human acid sphingomyelinase (hSMPD1) in a cell.

A recombinant AAV (rAAV) comprising an AAV capsid and a vector genome packaged therein is provided. In certain embodiments, the vector genome comprises an engineered nucleic acid sequence encoding a human acid sphingomyelinase (hSMPD1) operably linked to a regulatory sequence which directs expression of the hSMPD1, and an AAV 3′ ITR, wherein the hSMPD1 coding sequence is SEQ ID NO: 22 or a coding sequence at least 90% identical to SEQ ID NO: 22 which encodes the hSMPD1 of SEQ ID NO:2 (also reproduced in SEQ ID NO: 23), SEQ ID NO: 3 or a coding sequence at least 90% identical to SEQ ID NO: 3 which encodes the hSMPD1 of SEQ ID NO:2 (also reproduced in SEQ ID NO: 23). In certain embodiments, the hSMPD1 coding sequence encodes the hSMD1 having an Ala at position 36 and/or a G at position 506 of the human sequence, which reference to the number of SEQ ID NO: 2 (or 23). In certain embodiments, the hSMPD1 protein has the sequence of SEQ ID NO: 2 (or 23) or SEQ ID NO: 3. In other embodiments, the hSMPD1 protein comprises an exogenous leader sequence fused to an hSMPD1 protein comprising amino acids 47 to 631 of SEQ ID NO: 2 (SEQ ID NO: 23) or SEQ ID NO: 3. The regulatory sequences may comprise a UbC or a CB7 promoter and may comprise an SV40 late or a rabbit beta globin polyadenylation site. In certain embodiments, the AAV vector genome comprises a CB7 promoter, an intron, the hSMPD1 coding sequence, and a rabbit beta globin sequence. In certain embodiments, the vector genome comprises the sequence of SEQ ID NO: 21, 19, 10, or 8, or comprises the sequence of SEQ ID NO: 20, 18, 11, or 9. In certain embodiments, the AAV vector genome comprises a UbC promoter, the hSMPD1 coding sequence, and an SV40 late polyadenylation sequence. In certain embodiments, the AAV vector genome further comprises full-length AAV2 inverted terminal repeat (ITR) sequences. In certain embodiments, the AAV capsid is an AAVhu68 capsid.

In certain embodiments, a pharmaceutical composition comprising a formulation buffer and a population of rAAV as described herein is provided. In certain embodiments, the pharmaceutical composition is suitable for co-therapy with a functional hSMPD1 protein.

In certain embodiments, the pharmaceutical composition is formulated for delivery via intracerebroventricular (ICV), intrathecal (IT), intracisternal or intravenous (IV) injection.

In certain embodiments, rAAV and compositions are provided for use in the treatment of Niemann Pick A and/or improving the symptoms thereof by mitigating weight loss or cachexia, mitigating loss of motor function, mitigating loss of cognitive function and prolonging survival.

Other aspects and advantages of the invention will be readily apparent from the following detailed description of the invention.

### BRIEF DESCRIPTION OF THE DRAWINGS

FIGS. 1A and 1B provide bar graphs illustrating the amount of sphingomyelin stored in liver and brain across multiple lines of Smpd1 knock-out (KO) mice. KO mice from Lines A, B and C showed a significant amount of sphingomyelin stored in liver (FIG. 1A) and brain (FIG. 1B) compared to wild-type (WT) mice.

FIG. 2A-2D depict cholesterol storage in the brain as indicated by the positive staining for filipin. (A) Smpd1 WT mice displayed no presence of cholesterol in the brain, whereas Smpd1 KO mice displayed an abundant amount of cholesterol stored in the brain (FIG. 2B). This amount was reduced in Smpd1 KO mice that were given ICV gene therapy (FIG. 2C). FIG. 2D shows positive staining for filipin quantified for WT, KO and KO+ICV AAV mice.

FIG. 3A is a graph illustrating latency to fall during rotarod performance in WT, KO and KO+AAV-treated mice. Smpd1 KO mice receiving PBS displayed a steady decrease in latency to fall over time compared to WT mice. Smpd1 KO mice receiving AAV. CB7.hSMPD1 or AAV.Ubc.hSMPD1 gene therapy displayed a rescue phenotype in the behavioral defect shown in Smpd1 KO mice. There was no difference between the CB7 and Ubc promoters. FIG. 3B shows the average purkinje neuron (PN) density per Lobule for AAV.CB7.SMPD1 and AAV.UbChSMPD1, compared to untreated wild-type (WT) mice and knock-out mice injected with phosphate buffered saline (PBS).

FIG. 4A depicts histochemical staining of cholesterol storage in the brain and macrophages in the liver, spleen and lungs. In column 2, Smpd1 KO mice displayed an abundant amount of cholesterol in the brain as well as lipid-filled macrophages in the liver, spleen and lungs compared to WT mice (Column 1). In Columns 3 and 4, respectively, Smpd1 KO mice receiving AAV.CB7.hSMPD1 or AAV.Ubc.hSMPD1 gene therapy ICV exhibited a reduction in cholesterol storage in the brain as well as an absence or decrease in lipid-filled macrophages in the liver, spleen and lungs. FIG. 4B illustrates macrophages in alveoli in SMPD knock-out mice. FIG. 4C shows the absence of lipid-laden enlarged macrophages in alveoli (in lung tissue) from treated mice, suggesting correction of CNS and peripheral disease.

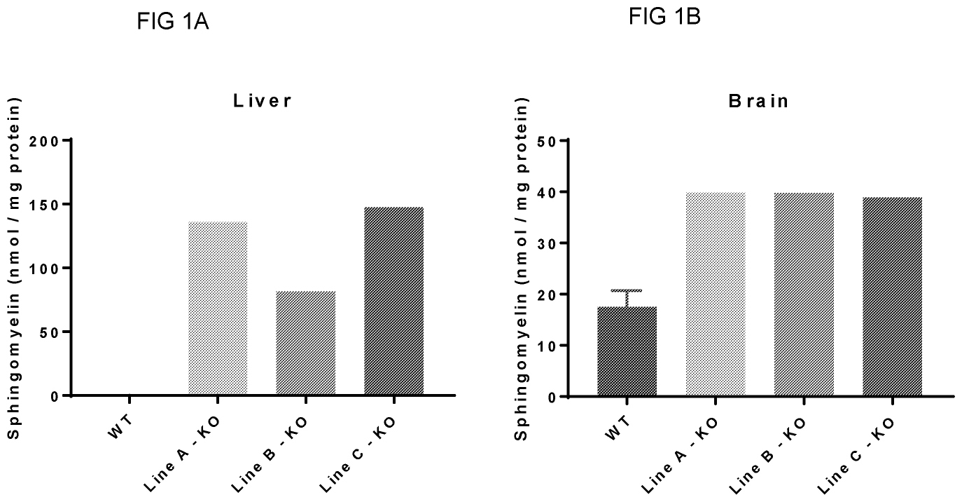

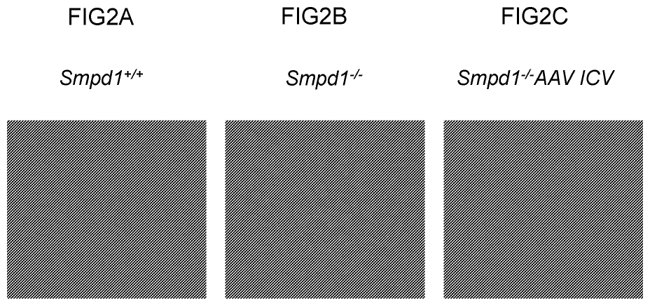

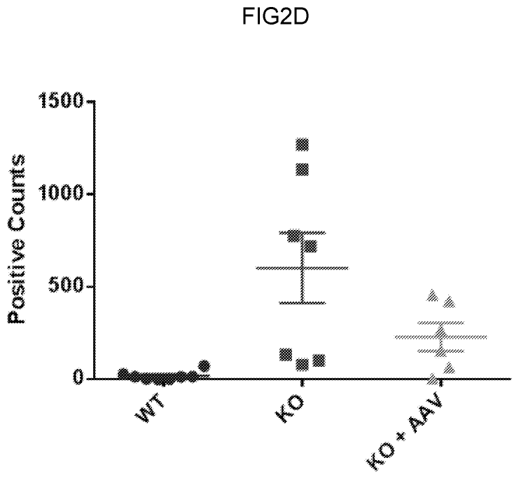

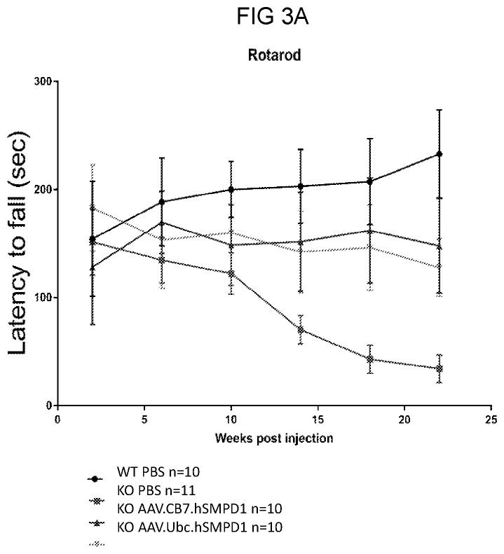

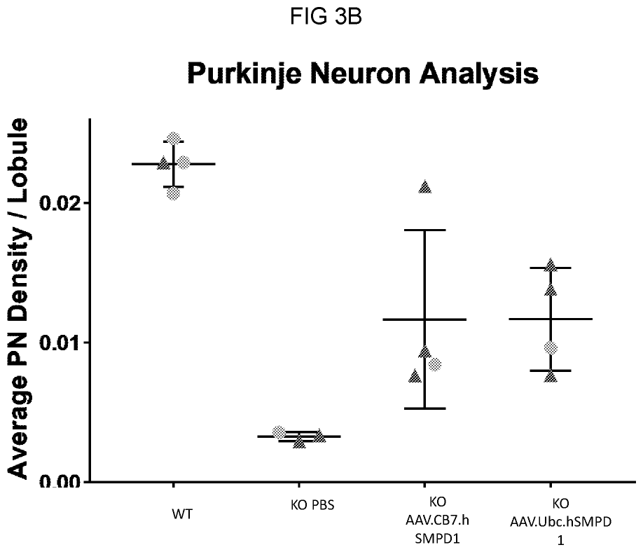

### DETAILED DESCRIPTION OF THE INVENTION

In certain embodiments, the compositions and methods described herein involve nucleic acid sequences, expression cassettes, vectors, recombinant viruses, and other compositions and methods for expression of a functional hSMPD1. In certain embodiments, the compositions and methods described herein involve nucleic acid sequences, expression cassettes, vectors, recombinant viruses, host cells, other compositions and methods for production of a composition comprising either a nucleic acid sequence encoding a functional hSMPD1 or a hSMPD1 polypeptide. In yet another embodiment, the compositions and methods described herein involve nucleic acid sequences, expression cassettes, vector genomes, vectors, recombinant viruses, other compositions and methods for delivery of the nucleic acid sequence encoding a functional hSMPD1 to a subject for the treatment of NPA disease. In certain embodiments, an adeno-associated viral (AAV) vector-based method described herein provides a new treatment option, helping to restore a desired function of hSMPD1 and to alleviate symptoms associated with hSMPD1-deficiency (NPA disease) by providing expression of a hSmpd1 in a subject in need thereof.

As used herein, the term “a therapeutic level” means a SMPD1 (ASMase) enzyme activity at least about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, about 100%, more than 100% of a healthy control.

Suitable assays for measuring hSMPD1 enzymatic activity are known to those of skill in the art. Additionally or alternatively, the function of the AAV-mediated delivery of the SMPD1 enzyme may be assessed by measuring reduction in lipid accumulation in cells, e.g., in brain, lung, spleen and/or liver. In some embodiments, such therapeutic levels of hSmpd1 may result in alleviation of NPA disease-related symptoms; improvement of NPA disease-related biomarkers of disease; reversal of certain NPA disease-related symptoms and/or prevention of progression of NPA disease-related symptoms; or any combination thereof. Treatment with vectors and compositions provided herein may provide one or more of behavioral rescue (correction), reduction of cholesterol storage in brain, and/or decreased or absent lipid-filled macrophages in liver, spleen and lungs.

As used herein, “disease,” “disorder,” and “condition” refer to NPA disease and/or hSMPD1 (ASMase) deficiency in a subject.

As used herein, the term “Niemann Pick A symptom(s)” or “NPA”, “symptom(s)” refers to symptom(s) found in patients with NPA disease as well as in animal models for NPA disease. Such symptoms include but are not limited to weight loss or cachexia, loss of motor function, lipid accumulation (e.g., in spleen, liver, lungs, bone marrow, and/or brain), enlarged spleen, enlarged liver, progressive deterioration of the nervous system, loss of cognitive function and premature death.

The vectors provided herein are well suited for administering to the mammal's CNS an effective amount of a viral vector comprising a transgene encoding acid sphingomyelinase polypeptide after administration of an effective amount of a viral vector comprising said transgene to the mammal's liver tissue. In another embodiment, the area of the CNS to which the viral vector is delivered is the brain.

In a yet further aspect, a method is provided to treat Niemann-Pick Type A disease (NPA) in a mammal suffering from NPA by administering an effective amount of an AAV viral vector comprising a transgene encoding an acid sphingomyelinase polypeptide (e.g., hSMPD1) to the mammal's liver tissue and subsequently delivering an effective amount of an AAV vector comprising a transgene encoding an acid sphingomyelinase polypeptide to the mammal's CNS, thereby treating NPA in the mammal. In another embodiment, the area of the CNS to which the viral vector is delivered is the brain.

1. Human Acid Sphingomyelin Phosphodiester 1 (hSmpd1)

The SMPD1 gene encodes the SMPD1 protein, which has enzymatic activity and which is also known as acid sphingomyelinase (or ASMase). A “functional SMPD1 protein” or a “functional ASMase” (also referenced to as human SMPD1 or hSMPD1) as used herein is capable of converting the lipid sphingomyelin into ceramide, so that fat does not accumulate in cells. Loss of SMPD1 enzymatic function is associated with cell lose and impaired function of tissues and organs including the brain, lungs, spleen, and liver in people with Niemann-Pick disease type A.

As used herein, the term “functional hSmpd1” refers to a SMPD1 enzyme which provides at least about 10%, at least about 20%, at least about 30%, at least about 40%, at least about 50%, at least about 60%, at least about 70%, at least about 75%, at least about 80%, at least about 90%, or about the same, or greater than 100% of the biological activity level of a native (wild-type) hSMPD1 from a patient without NPA.

The hSMPD1 may be, for example, a full-length protein (including a signal peptide and the mature protein), the mature protein, a mature protein with a short native signal peptide as described herein (e.g., 629 amino acids), a mature protein with a full-length native signal peptide (e.g., 631 amino acids), a mature with an exogenous signal peptide, or a functional fragment.

As used herein, a “signal peptide” refers to a short peptide present at the N-terminus of newly synthesized proteins. A signal peptide, and in some cases the nucleic acid sequences encoding such a peptide, may also be referred to as a signal sequence, a targeting signal, a localization signal, a localization sequence, a transit peptide, a leader sequence, or a leader peptide.

As described herein, an hSMPD1 may include a native signal peptide (i.e., amino acids 1 to 46 of SEQ ID NO: 2 or SEQ ID NO: 30) or, alternatively, an exogenous signal peptide. With reference to the numbering of the hSMPD1 of SEQ ID NO: 2, there is a signal peptide (about amino acid positions 1 to 46 of SEQ ID NO: 2 or SEQ ID NO: 30) and the mature protein comprises about amino acid 47 to about 629 of SEQ ID NO: 2 (or SEQ ID NO: 31). The amino acid sequence of SEQ ID NO: 2 is also reproduced in SEQ ID NO: 23.

In certain embodiments, a hSMPD1 includes a signal peptide from an exogenous source protein. Since certain NPA are associated with a longer signal peptide, the selected signal peptide is generally selected to be of the same length as provided in the constructs herein (e.g., about 46 amino acids, but may be selected to be shorter, e.g., about 10 to about 25 amino acids in length). In certain embodiments, such an exogenous signal peptide is preferably of human origin and may include, e.g., an IL-2 signal peptide. Other signal/leader peptides may be natively found in an immunoglobulin (e.g., IgG), a cytokine (e.g., IL-2, IL12, IL18, or the like), insulin, albumin, β-glucuronidase, alkaline protease or the fibronectin secretory signal peptides, amongst others. See, also, e.g., signalpeptide.de/index.php?m=listspdb_mammalia. Such a chimeric hSmpd1 may have the exogenous leader in the place of the entire native signal peptide. Optionally, an N-terminal truncation of the hSmpd1 enzyme may lack only a portion of the signal peptide (e.g., a deletion of about 2 to about 25 amino acids, or values therebetween), the entire signal peptide, or a fragment longer than the signal peptide (e.g., up to about amino acids 46). Optionally, such an enzyme may contain a C-terminal truncation of about 5, 10, 15, or 20 amino acids in length.

In certain embodiments, a functional hSMPD1 may be selected which has a sequence that encodes a protein at least 95% identical, at least 97% identical, or at least 99% identical to the sequence (amino acids 1 to 629) of SEQ ID NO: 2. SEQ ID NO: 23 has the same amino acid sequence as SEQ ID NO: 2, but a different coding sequence. In certain embodiments, provided is a hSMPD1 sequence which encodes a protein at least 95%, at least 97%, or at least 99% identical to the mature protein (amino acids 47 to 629) of SEQ ID NO: 2 or 23. For example, in certain embodiments, the hSMPD1 protein has an Ala substitution at position 36 and/or a R substitution at position 506, with respect to the numbering in SEQ ID NO: 2 or 23.

In certain embodiments, a functional hSMPD1 may be selected which has a sequence that encodes a protein at least 95% identical, at least 97% identical, or at least 99% identical to the sequence (amino acids 1 to 629) of SEQ ID NO: 3. In certain embodiments, provided is a hSMPD1 sequence which encodes a protein at least 95%, at least 97%, or at least 99% identical to the mature protein (amino acids 47 to 629) of SEQ ID NO: 3. For example, in certain embodiments, the hSMPD1 protein has a Val at position 36 and/or a R substitution at position 506, with respect to the numbering in SEQ ID NO: 3.

As used herein, the “conservative amino acid replacement” or “conservative amino acid substitutions” refers to a change, replacement or substitution of an amino acid to a different amino acid with similar biochemical properties (e.g., charge, hydrophobicity and size), which is known by practitioners of the art. Also see, e.g., FRENCH et al. What is a conservative substitution? Journal of Molecular Evolution, March 1983, Volume 19, Issue 2, pp 171-175 and YAMPOLSKY et al. The Exchangeability of Amino Acids in Proteins, Genetics. 2005 August; 170(4): 1459-1472, each of which is incorporated herein by reference in its entirety.

In one aspect, provided herein are nucleic acid sequences and, for example, expressions cassettes and vectors comprising the same, which encode a functional hSMPD1 protein. In one embodiment, the nucleic acid sequence is the engineered hSMPD1 sequence of SEQ ID NO: 4, which encodes a hSMDP1 having an A at position 36 with reference to the numbering of SEQ ID NO:2 or 23, which is a 629 amino acid hSMPD1 protein (having a short leader sequence) having an Ala (A) in position 36 and Gly (G) in position 506. In certain, the nucleic acid sequence is at least about 80% identical to the engineered hSMPD1 sequence of SEQ ID NO: 4, which encodes a functional hSMPD1 having an Ala at position 36 with reference to the numbering of SEQ ID NO:2. In certain embodiments, the encoded protein is a 629 amino acid hSMPD1 protein having a native signal peptide. In other embodiments, the encoded protein is a 631 protein having the longer, native signal peptide. In certain embodiments, the encoded protein has an Arg (R) in position 506.

In another one aspect, provided herein are nucleic acid sequences and, for example expressions cassettes and vectors comprising the same, which encode a functional hSMPD1 protein. In one embodiment, the nucleic acid sequence is the engineered hSMPD1 sequence (hMPDD1.CoV2 or CoV2) of SEQ ID NO: 22, which encodes a hSMPD1 having an A at position 36 with reference to the numbering of SEQ ID NO: 2 and 23, which is a 629 amino acid hSMPD1 protein (having a short leader sequence) having an Ala (A) in position 36 and Gly (G) in position 506. In certain, the hSPMPD1.CoV2 nucleic acid sequence is at least about 80% identical to the engineered hSMPD1 sequence of SEQ ID NO: 22, which encodes a functional hSMPD1 having an Ala at position 36 with reference to the numbering of SEQ ID NO: 2 and 23. In certain embodiments, the encoded protein is a 629 amino acid hSMPD1 protein having a native signal peptide. In other embodiments, the encoded protein is a 631 protein having the longer, native signal peptide. In certain embodiments, the encoded protein has an Arg (R) in position 506.

In one aspect, provided herein are nucleic acid sequences and, for example expressions cassettes and vectors comprising the same, which encode a functional hSMPD1 protein. In one embodiment, the nucleic acid sequence is the engineered hSMPD1 sequence of SEQ ID NO: 5, which encodes a hSMPD1 having an V at position 36 with reference to the numbering of SEQ ID NO:3, which is a 629 amino acid hSMPD1 protein (having a short leader sequence) having an Val (V) in position 36 and Gly (G) in position 506. In certain embodiments, the nucleic acid sequence is at least about 80% identical to the engineered hSMPD1 sequence of SEQ ID NO: 5, which encodes a functional hSMPD1 having an Ala at position 36 with reference to the numbering of SEQ ID NO:3. In certain embodiments, the encoded protein is a 629 amino acid hSMPD1 protein having a native signal peptide. In other embodiments, the encoded protein is a 631 protein having the longer, native signal peptide. In certain embodiments, the encoded protein has an Arg® in position 506.

As used herein, “a nucleic acid” refers to a polymeric form of nucleotides and includes RNA, mRNA, cDNA, genomic DNA, peptide nucleic acid (PNA) and synthetic forms and mixed polymers of the above. A nucleotide refers to a ribonucleotide, deoxynucleotide or a modified form of either type of nucleotide (e.g., a peptide nucleic acid oligomer). The term also includes single- and double-stranded forms of DNA. The skilled person will appreciate that functional variants of these nucleic acid molecules are described herein. Functional variants are nucleic acid sequences that can be directly translated, using the standard genetic code, to provide an amino acid sequence identical to that translated from a parental nucleic acid molecule.

In certain embodiments, the nucleic acid molecules encoding a functional hSmpd1, and other constructs as described herein are useful in generating expression cassettes and vector genomes and may be engineered for expression in yeast cells, insect cells, or mammalian cells, such as human cells. Methods are known and have been described previously (e.g., WO 96/09378). A sequence is considered engineered if at least one non-preferred codon as compared to a wild-type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in kazusa.jp/codon. Preferably more than one non-preferred codon, preferably most or all non-preferred codons, are replaced by codons that are more preferred. Preferably the most frequently used codons in an organism are used in an engineered sequence. Replacement by preferred codons generally leads to higher expression. It will also be understood by a skilled person that numerous different nucleic acid molecules can encode the same polypeptide as a result of the degeneracy of the genetic code. It is also understood that skilled persons may, using routine techniques, make nucleotide substitutions that do not affect the amino acid sequence encoded by the nucleic acid molecules to reflect the codon usage of any particular host organism in which the polypeptides are to be expressed. Therefore, unless otherwise specified, a “nucleic acid sequence encoding an amino acid sequence” includes all nucleotide sequences that are degenerate versions of each other and that encode the same amino acid sequence. Nucleic acid sequences can be cloned using routine molecular biology techniques, or generated de novo by DNA synthesis, which can be performed using routine procedures by service companies having business in the field of DNA synthesis and/or molecular cloning (e.g., GeneArt, GenScript, Life Technologies, Eurofins).

In certain embodiments, the nucleic acids, expression cassettes, vector genomes described herein include a hSMPD1 coding sequence that is an engineered sequence. In certain embodiments, the engineered sequence is useful to improve production, transcription, expression, or safety in a subject. In certain embodiments, the engineered sequence is useful to increase efficacy of the resulting therapeutic compositions or treatment. In further embodiments, the engineered sequence is useful to increase the efficacy of the functional hSMPD1 protein being expressed, and may also permit a lower dose of a therapeutic reagent that delivers the functional hSMPD1. In certain embodiments, the engineered hSMPD1 coding sequence is characterized by improved translation rate as compared to a wild type hSMPD1 coding sequence.

Efficacy may be determined by a variety of suitable methods, including an improvement in lethargic behavior, decrease or stabilization of gait abnormalities, CNS/respiratory/cardiac involvement. For example, to assess motor coordination, animals may be given rotarod performance tests. Filipin immunostaining on brain sections may be used to detect cholesterol deposits. In certain embodiments, efficacy is determined by a decrease in the amount of stored cholesterol. Other assays for measuring efficacy may include sphingomyelin quantification (liver/brain/spleen) and a chitinase assay (biomarker of disease severity).

In certain embodiments, the subjects provided herein are monitored for DRG pathology via SNAP pathology and/or via use of a biomarker. See, e.g., U.S. Patent Application No. 63/279,561, filed Nov. 15, 2021, which is incorporated herein by reference.

By “engineered” is meant that the nucleic acid sequences encoding a functional hSMPD1 enzyme described herein are assembled and placed into any suitable genetic element, e.g., naked DNA, phage, transposon, cosmid, episome, etc., which transfers the hSmpd1 sequences carried thereon to a host cell, e.g., for generating non-viral delivery systems (e.g., RNA-based systems, naked DNA, or the like), or for generating viral vectors in a packaging host cell, and/or for delivery to a host cell in a subject. In certain embodiments, the genetic element is a vector. In one embodiment, the genetic element is a plasmid. The methods used to make such engineered constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of a construct, the full-length of a gene coding sequence, or a fragment of at least about 500 to 1000 nucleotides. However, identity among smaller fragments, for example, of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 100 amino acids, about 300 amino acids, or a peptide fragment thereof or the corresponding nucleic acid sequence coding sequences. A suitable amino acid fragment may be at least about 8 amino acids in length, and may be up to about 50 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Identity may be determined by preparing an alignment of sequences and through the use of a variety of algorithms and/or computer programs known in the art or commercially available (e.g., BLAST, ExPASy; Clustal Omega; FASTA; using, e.g., Needleman-Wunsch algorithm, Smith-Waterman algorithm). Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

### 2. Expression Cassettes

In certain embodiments, provided herein are expression cassettes having an engineered nucleic acid sequence encoding a functional hSmpd1 and a regulatory sequence which directs the expression thereof. In further embodiments, an expression cassette having an engineered nucleic acid sequence as described herein, which encodes a functional hSmpd1, and a regulatory sequence which directs the expression thereof.

As used herein, the term “expression” or “gene expression” refers to the process by which information from a gene is used in the synthesis of a functional gene product. The gene product may be a protein, a peptide, or a nucleic acid polymer (such as an RNA, a DNA or a PNA).

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences that are contiguous or non-contiguous with the nucleic acid sequence and regulatory sequences that act in trans or cis nucleic acid sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell.

Typically, such an expression cassette can be used for generating a viral vector and contains the coding sequence for the gene product described herein flanked by packaging signals of the viral genome and other expression control sequences such as those described herein. In certain embodiments, a vector genome may contain two or more expression cassettes.

In certain embodiments, the expression cassette refers to a nucleic acid polymer which comprises the coding sequences for a function hSMPD1 (including variants and fragments thereof) and a promoter. In further embodiments, the expression cassettes include one or more regulatory sequences in addition to a promoter. In certain embodiments, the expression vector is a vector genome. In certain embodiments, the expression cassette or vector genome is packaged into a vector. In certain embodiments, a plasmid that includes an expression cassette described herein is provided.

As used herein, the term “regulatory sequence” or “expression control sequence” refers to nucleic acid sequences, such as initiator sequences, enhancer sequences, and promoter sequences, which induce, repress, or otherwise control the transcription of protein encoding nucleic acid sequences to which they are operably linked.

As used herein, the term “operably linked” refers to both expression control sequences that are contiguous with the nucleic acid sequence encoding the hSmpd1 and/or expression control sequences that act in trans or at a distance to control the transcription and expression thereof.

The term “heterologous” when used with reference to a protein or a nucleic acid in a plasmid, expression cassette, or vector, indicates that the protein or the nucleic acid is present with another sequence or subsequence which with which the protein or nucleic acid in question is not found in the same relationship to each other in nature.

In certain embodiments, the expression cassette provided includes a promoter that is a chicken β-actin promoter. A variety of chicken beta-actin promoters have been described alone, or in combination with various enhancer elements (e.g., CB7 (also referred to as CB7 hybrid promoter) is a chicken beta-actin promoter with cytomegalovirus (CMV IE) enhancer elements, a CAG promoter, which includes the promoter, the first exon and first intron of chicken beta actin, and the splice acceptor of the rabbit beta-globin gene), or a CBh promoter [S J Gray et al, Hu Gene Ther, 2011 Sep.; 22(9): 1143-1153]. In certain embodiments, the expression cassette includes a promoter that is a ubiquitin promoter, UbC.

In other embodiments, a suitable promoter may include without limitation, an elongation factor 1 alpha (EF1 alpha) promoter (see, e.g., Kim D W et al, Use of the human elongation factor 1 alpha promoter as a versatile and efficient expression system. Gene. 1990 Jul. 16; 91(2):217-23), a Synapsin 1 promoter (see, e.g., Kugler S et al, Human synapsin 1 gene promoter confers highly neuron-specific long-term transgene expression from an adenoviral vector in the adult rat brain depending on the transduced area. Gene Ther. 2003 Feb.; 10(4):337-47), a neuron-specific enolase (NSE) promoter (see, e.g., Kim J et al, Involvement of cholesterol-rich lipid rafts in interleukin-6-induced neuroendocrine differentiation of LNCaP prostate cancer cells. Endocrinology. 2004 February; 145(2):613-9. Epub 2003 Oct. 16), or a CB6 promoter (see, e.g., Large-Scale Production of Adeno-Associated Viral Vector Serotype-9 Carrying the Human Survival Motor Neuron Gene, Mol Biotechnol. 2016 January; 58(1):30-6. doi: 10.1007/s12033-015-9899-5).

Examples of promoters that are tissue-specific are well known for liver and other tissues (albumin, Miyatake et al., (1997) J. Virol., 71:5124-32; hepatitis B virus core promoter, Sandig et al., (1996) Gene Ther., 3:1002-9; alpha-fetoprotein (AFP), Arbuthnot et al., (1996) Hum. Gene Ther., 7:1503-14), bone osteocalcin (Stein et al., (1997) Mol. Biol. Rep., 24:185-96); bone sialoprotein (Chen et al., (1996) J. Bone Miner. Res., 11:654-64), lymphocytes (CD2, Hansal et al., (1998) J. Immunol., 161:1063-8; immunoglobulin heavy chain; T cell receptor chain), neuronal such as neuron-specific enolase (NSE) promoter (Andersen et al., (1993) Cell. Mol. Neurobiol., 13:503-15), neurofilament light-chain gene (Piccioli et al., (1991) Proc. Natl. Acad. Sci. USA, 88:5611-5), and the neuron-specific vgf gene (Piccioli et al., (1995) Neuron, 15:373-84), among others. In certain embodiments, the promoter is a human thyroxine binding globulin (TBG) promoter. Alternatively, a regulatable promoter may be selected. See, e.g., WO 2011/126808B2, incorporated by reference herein.

In certain embodiments, the expression cassette includes one or more expression enhancers. In certain embodiments, the expression cassette contains two or more expression enhancers. These enhancers may be the same or may be different. For example, an enhancer may include an Alpha mic/bik enhancer or a CMV enhancer (e.g., CMV IE enhancer). This enhancer may be present in two copies which are located adjacent to one another. Alternatively, the dual copies of the enhancer may be separated by one or more sequences. In still further embodiments, the expression cassette further contains an intron, e.g., a chicken beta-actin intron, a human 0-globulin intron, SV40 intron, and/or a commercially available Promega® intron (i.e., Promega chimeric intron. Other suitable introns include those known in the art, e.g., such as are described in WO 2011/126808.

The expression cassettes provided may include one or more expression enhancers such as post-transcriptional regulatory element from hepatitis viruses of woodchuck (WPRE), human (HPRE), ground squirrel (GPRE) or arctic ground squirrel (AGSPRE); or a synthetic post-transcriptional regulatory element. These expression-enhancing elements are particularly advantageous when placed in a 3′ UTR and can significantly increase mRNA stability and/or protein yield. In certain embodiments, the expression cassettes provided include a regulator sequence that is a woodchuck hepatitis virus posttranscriptional regulatory element (WPRE) or a variant thereof. Suitable WPRE sequences are provided in the vector genomes described herein and are known in the art (e.g., such as those are described in U.S. Pat. Nos. 6,136,597, 6,287,814, and 7,419,829, which are incorporated by reference). In certain embodiments, the WPRE is a modified WPRE sequence, which may be engineered upstream of the polyA sequence and downstream of the coding sequence [see, e.g., MA Zanta-Boussif, et al, Gene Therapy (2009) 16: 605-619, which is incorporated herein by reference].

Further, expression cassettes provided include a suitable polyadenylation signal. In certain embodiments, the polyA sequence is a rabbit globin poly A (also referred to as rabbit beta globin polyA or RBG polyA). See, e.g., WO 2014/151341. In another embodiment, the polyA sequence is a bovine growth hormone polyA. Alternatively, another polyA, e.g., a human growth hormone (hGH) polyadenylation sequence, an SV50 polyA, or a synthetic polyA is included.

In certain embodiments, the expression cassette may include one or more miRNA (also referred to as miR or micro-RNA) target sequences in the untranslated region(s). The miRNA target sequences are designed to be specifically recognized by miRNA present in cells in which transgene expression is undesirable and/or reduced levels of transgene expression are desired. In certain embodiments, the expression cassette includes miRNA target sequences that specifically reduce expression of hSMPD1 in dorsal root ganglion. In certain embodiments, the miRNA target sequences are located in the 3′ UTR, 5′ UTR, and/or in both 3′ and 5′ UTR of an expression cassette. In certain embodiments, the expression cassette comprises at least two tandem repeats of dorsal root ganglion (DRG)-specific miRNA target sequences, wherein the at least two tandem repeats comprise at least a first miRNA target sequence and at least a second miRNA target sequence which may be the same or different. In certain embodiments, the start of the first of the at least two drug-specific miRNA tandem repeats is within 20 nucleotides from the 5′ or 3′ end of the hSmpd1-coding sequence. In certain embodiments, the start of the first of the at least two DRG-specific miRNA tandem repeats is at least 100 nucleotides from the 5′ or 3′ end of the hSMPD1-coding sequence. In certain embodiments, the miRNA tandem repeats comprise 200 to 1200 nucleotides in length. Illustrative DRG targeting sequences (e.g., miR182) are provided in SEQ ID NO: 24, and a 4× tandem repeat is provided in SEQ ID NO: 28. In certain embodiments, the inclusion of miR targets does not modify the expression or efficacy of the therapeutic transgene in one or more target tissues, relative to the expression cassette lacking the miR target sequences. In certain embodiments, the miR is miR183. See, International Patent Application No. PCT/US19/67872, filed Dec. 20, 2019 and published as WO 202/132455, Jun. 25, 2020; International Patent Application No. PCT/US2021/032003, filed 12 May 2021 (claiming priority to U.S. Provisional Patent Application No. 63/023,594, filed May 12, 2020, U.S. Provisional Patent Application No. 63/038,488, filed Jun. 12, 2020, U.S. Provisional Patent Application No. 63/043,562, filed Jun. 24, 2020, and U.S. Provisional Patent Application No. 63/079,299, filed Sep. 16, 2020, all of which are incorporated by reference in their entireties), and now published as WO 2021/231579, published Nov. 18, 2021.

Examples of illustrative expression cassettes are provided in the examples below and include, e.g., CB7.CI.hSMPD1co(TY).RBG, comprising a CB7 hybrid promoter (comprising a CMV IE enhancer and CB promoter), a chicken beta actin intron, hSMPD1.V36A.co (SEQ ID NO: 4), and a Rabbit beta globin polyA (SEQ ID NO: 8). In another embodiment, an expression cassette is comprises a CB7 hybrid promoter, a chicken beta actin intron, the hSMPD1cov2 coding sequence of SEQ ID NO: 22), and a rabbit beta globin polyA [CB7.CI.hSMPD1cov2.RBG (SEQ ID NO: 21). In still another embodiment, an expression cassette comprises a CB7 hybrid promoter, a chicken beta actin intron, the hSMPD1cov2 coding sequence of SEQ ID NO: 22, 4× miR [SEQ ID NO: 28, which comprises four copies of SEQ ID NO: 24], and a rabbit beta globin polyA [CB7.CI.hSMPD1coV2.4xmiR182.rBG (SEQ ID NO: 19)].

It should be understood that the compositions in these and other expression cassettes described are intended to be applied to other compositions, regimens, aspects, embodiments and methods described across the Specification.

### 3. Production of Delivery Vectors

In one aspect, provided herein is a vector comprising a nucleic acid sequence encoding a functional hSMPD1. In certain embodiments, the vector comprises an expression cassette as described herein for delivery of a hSmpd1 coding sequence.

A “vector” as used herein is a biological or chemical moiety comprising a nucleic acid sequence which can be introduced into an appropriate target cell for replication or expression of said nucleic acid sequence. Examples of a vector include but not limited to a recombinant virus, a plasmid, Lipoplexes, a Polymersome, Polyplexes, a dendrimer, a cell penetrating peptide (CPP) conjugate, a magnetic particle, or a nanoparticle. In certain embodiments, a vector is a nucleic acid molecule into which an engineered nucleic acid encoding a functional hSMPD1 may be inserted, which can then be introduced into an appropriate target cell. Such vectors preferably have one or more origin of replication, and one or more site into which the recombinant DNA can be inserted. Vectors often have means by which cells with vectors can be selected from those without, e.g., they encode drug resistance genes. Common vectors include plasmids, viral genomes, and “artificial chromosomes”. Conventional methods of generation, production, characterization or quantification of the vectors are available to one of skill in the art.

In certain embodiments, the vector is a non-viral plasmid that comprises an expression cassette described herein (for example, “naked DNA”, “naked plasmid DNA”, RNA, and mRNA, which may be coupled with various compositions and nano particles, including, for examples, micelles, liposomes, cationic lipid—nucleic acid compositions, poly-glycan compositions and other polymers, lipid and/or cholesterol-based—nucleic acid conjugates) and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference.

In certain embodiments, the vector described herein is a “replication-defective virus” or a “viral vector” which refers to a synthetic or artificial viral particle in which an expression cassette containing a nucleic acid sequence encoding hSMPD1 is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the nucleic acid sequence encoding hSMPD1 flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

As used herein, a recombinant virus vector is an adeno-associated virus (AAV), an adenovirus, a bocavirus, a hybrid AAV/bocavirus, a herpes simplex virus or a lentivirus.

In certain embodiments, a host cell having a nucleic acid including an hSMPD1-coding sequence is provided. In certain embodiments, the host cell contains a plasmid having an hSMPD1-coding sequence as described herein.

As used herein, the term “host cell” may refer to the packaging cell line in which a vector (e.g., a recombinant AAV) is produced. A host cell may be a prokaryotic or eukaryotic cell (e.g., human, insect, or yeast) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. Examples of host cells may include, but are not limited to an isolated cell, a cell culture, an Escherichia coli cell, a yeast cell, a human cell, a non-human cell, a mammalian cell, a non-mammalian cell, an insect cell, an HEK-293 cell, a liver cell, a kidney cell, a cell of the central nervous system, a neuron, a glial cell, or a stem cell.

In certain embodiments, a host cell contains an expression cassette for production of hSMPD1 such that the protein is produced in sufficient quantities in vitro for isolation or purification. In certain embodiments, the host cell contains an expression cassette encoding hSMPD1 (including, for example, a functional fragment thereof). As provided herein, hSMPD1 polypeptide may be included in a pharmaceutical composition administered to a subject as a therapeutic (i.e., enzyme replacement therapy).

As used herein, the term “target cell” refers to any cell in which expression of the functional hSmpd1 is desired. In certain embodiments, the term “target cell” is intended to reference the cells of the subject being treated for NPA disease. Examples of target cells may include, but are not limited to, liver cells, kidney cells, smooth muscle cells, and neurons. In certain embodiments, the vector is delivered to a target cell ex vivo. In certain embodiments, the vector is delivered to the target cell in vivo.

It should be understood that the compositions in the vector described herein are intended to be applied to other compositions, regiments, aspects, embodiments, and methods described across the Specification.

4. Recombinant Adeno-Associated Virus (rAAV)

In certain embodiments, provided herein is a rAAV comprising an AAV capsid and a vector genome packaged therein. The vector genome comprises an AAV 5′ inverted terminal repeat (ITR), a nucleic acid sequence encoding a functional hSMPD1 as described herein, a regulatory sequence which directs expression of hSMPD1 in a target cell, and an AAV 3′ ITR. In certain embodiments, the vector genome comprises an expression cassette as provided herein flanked by an AAV 5′ ITR and an AAV 3′ ITR. Such rAAV are suitable for use in the treatment of NPA disease.

As used herein, a “rAAV.hSmpd1” refers to a rAAV having a vector genome that includes a hSMPD1 coding sequence. A “rAAVhu68.hSMPD1” refers to rAAV having an AAVhu68 capsid and a vector genome that includes a hSMPD1 coding sequence.

As used herein, a “vector genome” refers to a nucleic acid sequence packaged inside a vector. In one embodiment, the vector genome refers to the nucleic acid sequence packaged inside a rAAV capsid forming an rAAV vector. Such a nucleic acid sequence contains AAV inverted terminal repeat sequences (ITRs). In certain embodiments, the ITRs are from an AAV different than that supplying a capsid. In a preferred embodiment, the ITR sequences from AAV2, or the deleted version thereof (AITR), which may be used for convenience and to accelerate regulatory approval. However, ITRs from other AAV sources may be selected.

Where the source of the ITRs is from AAV2 and the AAV capsid is from another AAV source, the resulting vector may be termed pseudotyped. In certain embodiments, the vector genome includes a shortened AAV2 ITR of 130 base pairs, wherein the external A elements is deleted. Without wishing to be bound by theory, it is believed that the shortened ITR reverts back to the wild-type length of 145 base pairs during vector DNA amplification using the internal (A′) element as a template. In other embodiments, full-length AAV 5′ and 3′ ITRs are used. Typically, AAV vector genome comprises an AAV 5′ ITR, regulatory sequence(s), a hSMPD1 coding sequence, and an AAV 3′ ITR. However, other configurations of these elements may be suitable. A shortened version of the 5′ ITR, termed AITR, has been described in which the D-sequence and terminal resolution site (trs) are deleted. In other embodiments, the full-length AAV 5′ and 3′ ITRs are used. In certain embodiments, the vector genome includes one or more miRNA target sequences.

In certain embodiments, a rAAV is provided having a vector genome that includes a 5′ ITR, a promoter, a chicken beta-actin intron, a hSMPD1 coding sequence, a poly A sequence, and a 3′ ITR. In certain embodiments, a rAAV is provided having a vector genome that includes a 5′ ITR, a CB7 hybrid promoter, a chicken beta-actin intron, a hSMPD1 coding sequence, and a rabbit globin poly A sequence, and a 3′ ITR. In certain embodiments, a rAAV is provided having a vector genome that includes a 5′ ITR, a TBG promoter, a chicken beta-actin intron, a hSMPD1 coding sequence, a WPRE, a bovine growth hormone poly A sequence, and a 3′ ITR. In certain embodiments, a rAAV is provided having a vector genome that includes a 5′ ITR, a UbC promoter, a hSMPD1 coding sequence, and a SV40 poly A sequence, and a 3′ ITR.

In certain embodiments, a rAAV is provided having a vector genome comprising a hSMPD1 coding sequence of SEQ ID NO: 22 or a sequence at least 80% identical, at least 85% identical, at least 90% identical, at least 95% identical, or at least 99 to 100% identical to SEQ ID NO: 22, which encode a functional hSMPD1 protein of SEQ ID NO: 2 and 23. In certain embodiments, a rAAV is provided having a vector genome comprising a hSMPD1 coding sequence of SEQ ID NO: 4 or a sequence at least 80% identical, at least 85% identical, at least 90% identical, at least 95% identical, or at least 99 to 100% identical to SEQ ID NO: 4, which encode a functional hSMPD1 protein. In certain embodiments, a rAAV is provided having a vector genome comprising a hSMPD1 coding sequence of SEQ ID NO: 5 or a sequence at least 80% identical, at least 85% identical, at least 90% identical, at least 95% identical, or at least 99 to 100% identical to SEQ ID NO: 5, which encode a functional hSMPD1 protein. In certain embodiments, a rAAV is provided having a vector genome comprising an expression cassette of SEQ ID NO: 8, 10, or 14. In certain embodiments, a rAAV is provided comprising a vector genome of SEQ ID NO: 9, 11, 14 or 15. In certain embodiments, an rAAV is provided comprising a vector genome of SEQ ID NO: 18. In certain embodiments, an rAAV is provided comprising a vector genome of SEQ ID NO: 20.

Examples of illustrative vector genome are provided in the examples below and include, e.g., ITR.CB7.CI.hSMPD1co(TY).RBG.ITR, comprising a CB7 hybrid promoter (comprising a CMV IE enhancer and CB promoter), a chicken beta actin intron, hSMPD1.V36A.co (SEQ ID NO: 4), and a Rabbit beta globin polyA (SEQ ID NO: 8), flanked at the 5′ end by a 5′ AAV ITR an spacer sequences, and at its 3′ end by spacer sequences and a 3′ ITR. See, e.g., SEQ ID NO: 9, optionally further comprising full-length 5′ and 3′ ITRs. In another embodiment, a vector genome comprises a CB7 hybrid promoter, a chicken beta actin intron, the hSMPD1cov2 coding sequence of SEQ ID NO: 22), and a rabbit beta globin polyA [ITR.CB7.CI.hSMPD1cov2.RBG.ITR (SEQ ID NO: 20). SEQ ID NO: 20 comprises the expression cassette of SEQ ID NO: 21, flanked at the 5′ end by a 5′ AAV ITR an spacer sequences, and at its 3′ end by spacer sequences and a 3′ ITR. In still another embodiment, a vector genome comprises a 5′ ITR, a spacer sequence, the expression cassette of SEQ ID NO: 19 (CB7.CI.hSMPD1coV2.4xmiR182.rBG), a spacer sequence, and a 3′ ITR. In certain embodiments, e.g., when packaged in an rAAV capsid (viral particle), the 5′ ITR and the 3′ ITR are full-length sequences. The expression cassette of SEQ ID NO: 19 comprises a CB7 hybrid promoter, a chicken beta actin intron, the hSMPD1cov2 coding sequence of SEQ ID NO: 22, 4×miR [SEQ ID NO: 28, which comprises four copies of SEQ ID NO: 24], and a rabbit beta globin polyA [CB7.CI.hSMPD1coV2.4xmiR182.rBG (SEQ ID NO: 19)].

In certain embodiments, pharmaceutical compositions comprising these rAAV.hSMPD1 are provided. In certain embodiments, these rAAV have AAVhu68 capsids. The encoded (predicated) amino acid sequence of the AAVhu68 VP1 capsid is reproduced in SEQ ID NO: 17. AAVhu68 coding sequence useful in production of rAAV having hu68 capsids are provided in SEQ ID NO: 17 and SEQ ID NO: 29 (AAVhu68M191). See, e.g., International Patent Application No. PCT/US2021/055436, filed 18 Oct. 2021, and WO 2018/160582.

As used herein, the terms “rAAV” and “artificial AAV” used interchangeably, mean, without limitation, an AAV comprising a capsid protein and a vector genome packaged therein, wherein the vector genome comprising a nucleic acid heterologous to the AAV. In one embodiment, the capsid protein is a non-naturally occurring capsid. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV2/5 and AAV2/8 are exemplary pseudotyped vectors. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

The term “AAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV DNase-resistant particle having an AAV protein capsid into which is packaged expression cassette flanked by AAV inverted terminal repeat sequences (ITRs) for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10). These documents also describe other AAVs which may be selected for generating AAVs and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models. Unless otherwise specified, the AAV capsid, ITRs, and other selected AAV components described herein, may be readily selected from among any AAV, including, without limitation, the AAVs commonly identified as AAV1, AAV2, AAV3, AAV4, AAV5, AAV6, AAV7, AAV8, AAV9, AAV8 bp, AAV7M8 and AAVAnc80, AAVhu68, and variants of any of the known or mentioned AAVs or AAVs yet to be discovered or variants or mixtures thereof. An AAV9 capsid includes an rAAV having capsid proteins comprising an amino acid sequence which is 99% identical to AAS99264. See, also U.S. Pat. No. 7,906,111 and WO 2005/033321. rAAVs having a AAVhu68 capsid are described in, for example, WO 2018/160582, which is incorporated herein by reference. In certain embodiments, the capsid protein is designated by a number or a combination of numbers and letters following the term “AAV” in the name of the rAAV vector. See also PCT/US19/19804 and PCT/US19/19861, each entitled “Novel Adeno-Associated Virus (AAV) Vectors, AAV Vectors Having Reduced Capsid Deamidation And Uses Therefor” and filed Feb. 27, 2019, which are incorporated by reference herein in their entireties.

As used herein, relating to AAV, the term “variant” means any AAV sequence which is derived from a known AAV sequence, including those with a conservative amino acid replacement, and those sharing at least 70%, at least 75%, at least 80%, at least 85%, at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3). As used herein “AAV9 variants” include those described in, e.g., WO2016/049230, U.S. Pat. No. 8,927,514, US 2015/0344911, and U.S. Pat. No. 8,734,809.

In certain embodiments, the AAV capsid is selected from among natural and engineered clade F adeno-associated viruses. In certain embodiments, the rAAV provided herein comprises an AAVhu68 capsid. See, US Published Patent Application US2020/0056159, incorporated by reference herein. AAVhu68 is within clade F. AAVhu68 (SEQ ID NO: 21) varies from another Clade F virus AAV9 by two encoded amino acids at positions 67 and 157 of vp1. In contrast, other Clade F AAVs (AAV9, hu31, hu32) have an Ala at position 67 and an Ala at position 157. However, in other embodiments, an AAV capsid is selected from a different clade, e.g., clade A, B, C, D, or E, or from an AAV source outside of any of these clades.

A rAAVhu68 is composed of an AAVhu68 capsid and a vector genome. In one embodiment, a composition comprising rAAVhu68 comprises an assembly of a heterogeneous population of vp1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences. SEQ ID NO: 16 (and SEQ ID NO: 29, AAVhu68M191) encode the amino acid sequence of the AAVhu68 vp1 protein (SEQ ID NO: 17). The AAVhu68 capsid contains subpopulations within the vp1 proteins, within the vp2 proteins and within the vp3 proteins which have modifications from the predicted amino acid residues in SEQ ID NO: 17. These subpopulations include, at a minimum, certain deamidated asparagine (N or Asn) residues. For example, certain subpopulations comprise at least one, two, three or four highly deamidated asparagines (N) positions in asparagine-glycine pairs in SEQ ID NO: 17 and optionally further comprising other deamidated amino acids, wherein the deamidation results in an amino acid change and other optional modifications. The various combinations of these and other modifications are described herein.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

In certain embodiments, an AAVhu68 capsid is further characterized by one or more of the following. AAVhu68 capsid proteins that comprise: AAVhu68 vp1 proteins produced by expression from a nucleic acid sequence which encodes the predicted amino acid sequence of 1 to 736 of SEQ ID NO: 17, vp1 proteins produced from SEQ ID NO: 16 (or SEQ ID NO: 29), or vp1 proteins produced from a nucleic acid sequence at least 70% identical to SEQ ID NO: 16 (or SEQ ID NO: 29) which encodes the predicted amino acid sequence of 1 to 736 of SEQ ID NO: 17; AAVhu68 vp2 proteins produced by expression from a nucleic acid sequence which encodes the predicted amino acid sequence of at least about amino acids 138 to 736 of SEQ ID NO: 17 (or SEQ ID NO: 32), vp2 proteins produced from a sequence comprising at least nucleotides 412 to 2211 of SEQ ID NO: 16 or SEQ ID NO: 29 (or SEQ ID NO: 33 or 34), or vp2 proteins produced from a nucleic acid sequence at least 70% identical to at least nucleotides 412 to 2211 of SEQ ID NO: 16 or SEQ ID NO: 29 (or SEQ ID NO: 33 or 34) which encodes the predicted amino acid sequence of at least about amino acids 138 to 736 of SEQ ID NO: 17 (or SEQ ID NO: 32), and/or AAVhu68 vp3 proteins produced by expression from a nucleic acid sequence which encodes the predicted amino acid sequence of at least about amino acids 203 to 736 of SEQ ID NO: 17 (or SEQ ID NO: 35), vp3 proteins produced from a sequence comprising at least nucleotides 607 to 2211 of SEQ ID NO: 16 (or SEQ ID NO: 35), or vp3 proteins produced from a nucleic acid sequence at least 70% identical to at least nucleotides 607 to 2211 of SEQ ID NO: 16 or SEQ ID NO: 29 (or SEQ ID NO: 36 or 37) which encodes the predicted amino acid sequence of at least about amino acids 203 to 736 of SEQ ID NO: 17 (or SEQ ID NO: 35). In certain embodiments, an rAAVhu68 capsid comprises a heterogeneous population of vp1 proteins which are the product of a nucleic acid sequence encoding the amino acid sequence of SEQ ID NO: 17, wherein the vp1 proteins comprise a Glutamic acid (Glu) at position 67 and/or a valine (Val) at position 157; a heterogeneous population of vp2 proteins optionally comprising a valine (Val) at position 157; and a heterogeneous population of vp3 proteins. The AAVhu68 capsid contains at least one subpopulation in which at least 65% of asparagines (N) in asparagine-glycine pairs located at position 57 of the vp1 proteins and at least 70% of asparagines (N) in asparagine-glycine pairs at positions 329, 452 and/or 512 of the vp1, v2 and vp3 proteins are deamidated, based on the residue numbering of the amino acid sequence of SEQ ID NO: 17, wherein the deamidation results in an amino acid change.

As used herein, “encoded amino acid sequence” refers to the amino acid which is predicted based on the translation of a known DNA codon of a referenced nucleic acid sequence being translated to an amino acid. As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 June; 78 (10: 6381-6388, which identifies Clades A, B, C, D, E and F, GenBank Accession Numbers AY530553 to AY530629. See, also, WO 2005/033321.

Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, VA). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

In certain embodiments, the rAAV is a self-complementary AAV. “Self-complementary AAV” or scAAV refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

In certain embodiments, the rAAV is nuclease-resistant. Such nuclease may be a single nuclease, or mixtures of nucleases, and may be endonucleases or exonucleases. A nuclease-resistant rAAV indicates that the AAV capsid has fully assembled and protects these packaged genomic sequences from degradation (digestion) during nuclease incubation steps designed to remove contaminating nucleic acids which may be present from the production process. In many instances, the rAAV described herein is DNase resistant.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette as described herein flanked by AAV inverted terminal repeats (ITRs); and sufficient helper functions to permit packaging of the expression cassette into the AAV capsid protein. Also provided herein is the host cell which contains a nucleic acid sequence encoding an AAV capsid; a functional rep gene; a vector genome as described; and sufficient helper functions to permit packaging of the vector genome into the AAV capsid protein. In one embodiment, the host cell is a HEK 293 cell. These methods are described in more detail in WO2017160360 A2, which is incorporated by reference herein.

Other methods of producing rAAV available to one of skill in the art may be utilized. Suitable methods may include without limitation, baculovirus expression system or production via yeast. See, e.g., Robert M. Kotin, Large-scale recombinant adeno-associated virus production. Hum Mol Genet. 2011 Apr. 15; 20(R1): R2-R6. Published online 2011 Apr. 29. doi: 10.1093/hmg/ddr141; Aucoin M G et al., Production of adeno-associated viral vectors in insect cells using triple infection: optimization of baculovirus concentration ratios. Biotechnol Bioeng. 2006 Dec. 20; 95(6):1081-92; SAMI S. THAKUR, Production of Recombinant Adeno-associated viral vectors in yeast. Thesis presented to the Graduate School of the University of Florida, 2012; Kondratov 0 et al. Direct Head-to-Head Evaluation of Recombinant Adeno-associated Viral Vectors Manufactured in Human versus Insect Cells, Mol Ther. 2017 Aug. 10. pii: 51525-0016(17)30362-3. doi: 10.1016/j.ymthe.2017.08.003. [Epub ahead of print]; Mietzsch M et al, OneBac 2.0: Sf9 Cell Lines for Production of AAV1, AAV2, and AAV8 Vectors with Minimal Encapsidation of Foreign DNA. Hum Gene Ther Methods. 2017 Feb.; 28(1):15-22. doi: 10.1089/hgtb.2016.164.; Li L et al. Production and characterization of novel recombinant adeno-associated virus replicative-form genomes: a eukaryotic source of DNA for gene transfer. PLoS One. 2013 Aug. 1; 8(8):e69879. doi: 10.1371/journal.pone.0069879. Print 2013; Galibert L et al, Latest developments in the large-scale production of adeno-associated virus vectors in insect cells toward the treatment of neuromuscular diseases. J Inverter Pathol. 2011 July; 107 Suppl:S80-93. doi: 10.1016/j.jip.2011.05.008; and Kotin R M, Large-scale recombinant adeno-associated virus production. Hum Mol Genet. 2011 Apr. 15; 20(R1):R2-6. doi: 10.1093/hmg/ddr141. Epub 2011 Apr. 29.

A two-step affinity chromatography purification at high salt concentration followed by anion exchange resin chromatography are used to purify the vector drug product and to remove empty capsids. These methods are described in more detail in WO 2017/160360 entitled “Scalable Purification Method for AAV9”, which is incorporated by reference herein. In brief, the method for separating rAAV9 particles having packaged genomic sequences from genome-deficient AAV9 intermediates involves subjecting a suspension comprising recombinant AAV9 viral particles and AAV 9 capsid intermediates to fast performance liquid chromatography, wherein the AAV9 viral particles and AAV9 intermediates are bound to a strong anion exchange resin equilibrated at a pH of 10.2, and subjected to a salt gradient while monitoring eluate for ultraviolet absorbance at about 260 and about 280. Although less optimal for rAAV9, the pH may be in the range of about 10.0 to 10.4. In this method, the AAV9 full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2/9 serotype. Under these ionic conditions, a significant percentage of residual cellular DNA and proteins flow through the column, while AAV particles are efficiently captured.

Conventional methods for characterization or quantification of rAAV are available to one of skill in the art. To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles. Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

Methods for determining the ratio among vp1, vp2 and vp3 of capsid protein are also available. See, e.g., Vamseedhar Rayaprolu et al, Comparative Analysis of Adeno-Associated Virus Capsid Stability and Dynamics, J Virol. 2013 December; 87(24): 13150-13160; Buller R M, Rose J A. 1978. Characterization of adenovirus-associated virus-induced polypeptides in KB cells. J. Virol. 25:331-338; and Rose J A, Maizel J V, Inman J K, Shatkin A J. 1971. Structural proteins of adenovirus-associated viruses. J. Virol. 8:766-770.

As used herein, the term “treatment” or “treating” refers to composition(s) and/or method(s) for the purposes of amelioration of one or more symptoms of NPA disease, restore of a desired function of hSmpd1, or improvement of a biomarker of disease. In some embodiments, the term “treatment” or “treating” is defined encompassing administering to a subject one or more compositions described herein for the purposes indicated herein. “Treatment” can thus include one or more of reducing onset or progression of NPA disease, preventing disease, reducing the severity of the disease symptoms, retarding their progression, removing the disease symptoms, delaying progression of disease, or increasing efficacy of therapy in a given subject.

It should be understood that the compositions in the rAAV described herein are intended to be applied to other compositions, regiments, aspects, embodiments and methods described across the Specification.

### 5. Pharmaceutical Compositions or Formulations

In certain embodiments, provided herein is a pharmaceutical composition comprising a vector, such as a rAAV, as described herein in a formulation buffer. In certain embodiments, the pharmaceutical composition is suitable for co-administering with a functional hSmpd1 protein. In one embodiment, provided is a pharmaceutical composition comprising a rAAV as described herein in a formulation buffer. In certain embodiments, the rAAV is formulated at about 1×10⁹ genome copies (GC)/mL to about 1×10¹⁴ GC/mL. In a further embodiment, the rAAV is formulated at about 3×10⁹ GC/mL to about 3×10¹³ GC/mL. In yet a further embodiment, the rAAV is formulated at about 1×10⁹ GC/mL to about 1×10¹³ GC/mL. In one embodiment, the rAAV is formulated at least about 1×10¹¹ GC/mL.

In certain embodiments, the pharmaceutical composition comprises the expression cassette comprising a hSmpd1 coding sequence in a non-viral or viral vector system. This may include, e.g., naked DNA, naked RNA, an inorganic particle, a lipid or lipid-like particle, a chitosan-based formulation and others known in the art and described for example by Ramamoorth and Narvekar, as cited above). Such a non-viral vector system may include, e.g., a plasmid or non-viral genetic element, or a protein-based vector.

In certain embodiments, the pharmaceutical composition comprises a non-replicating viral vector. Suitable viral vectors may include any suitable delivery vector, such as, e.g., a recombinant adenovirus, a recombinant lentivirus, a recombinant bocavirus, a recombinant adeno-associated virus (AAV), or another recombinant parvovirus. In certain embodiments, the viral vector is a recombinant AAV for delivery of a hSmpd1 to a patient in need thereof.

In one embodiment, the pharmaceutical composition comprises a vector that includes an expression cassette comprising a hSmpd1 coding sequence, and a formulation buffer suitable for delivery via intracerebroventricular (ICV), intrathecal (IT), intracisternal or intravenous (IV) injection. In one embodiment, the expression cassette comprising the hSmpd1 coding sequence is in packaged a recombinant AAV.

In one embodiment, the pharmaceutical composition comprises a functional hSmpd1 polypeptide, or a functional fragment thereof, for delivery to a subject as an enzyme replacement therapy (ERT). Such pharmaceutical compositions are usually administered intravenously, however intradermal, intramuscular, or oral administration is also possible in some circumstances. The compositions can be administered for prophylactic treatment of individuals suffering from, or at risk of, NPA disease. For therapeutic applications, the pharmaceutical compositions are administered to a patient suffering from established disease in an amount sufficient to reduce the concentration of accumulated metabolite and/or prevent or arrest further accumulation of metabolite. For individuals at risk of lysosomal enzyme deficiency disease, the pharmaceutical compositions are administered prophylactically in an amount sufficient to either prevent or inhibit accumulation of metabolite. The pharmaceutical compositions comprising a hSmpd1 protein described herein are administered in a therapeutically effective amount. In general, a therapeutically effective amount can vary depending on the severity of the medical condition in the subject, as well as the subject's age, general condition, and gender. Dosages can be determined by the physician and can be adjusted as necessary to suit the effect of the observed treatment. In one aspect, provided herein is a pharmaceutical composition for ERT formulated to contain a unit dosage of a hSmpd1 protein, or functional fragment thereof.

In certain embodiments, the formulation further comprises a surfactant, preservative, excipients, and/or buffer dissolved in the aqueous suspending liquid. In one embodiment, the buffer is PBS. In another embodiment, the buffer is an artificial cerebrospinal fluid (aCSF), e.g., Eliott's formulation buffer; or Harvard apparatus perfusion fluid (an artificial CSF with final Ion Concentrations (in mM): Na 150; K 3.0; Ca 1.4; Mg 0.8; P 1.0; Cl 155). Various suitable solutions are known including those which include one or more of: buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration.

Suitably, the formulation is adjusted to a physiologically acceptable pH, e.g., in the range of pH 6 to 8, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous delivery, a pH of 6.8 to about 7.2 may be desired. However, other pHs within the broadest ranges and these subranges may be selected for other routes of delivery.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly (propylene oxide)) flanked by two hydrophilic chains of polyoxyethylene (poly (ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In one example, the formulation may contain, e.g., buffered saline solution comprising one or more of sodium chloride, sodium bicarbonate, dextrose, magnesium sulfate (e.g., magnesium sulfate −7H2O), potassium chloride, calcium chloride (e.g., calcium chloride −2H2O), dibasic sodium phosphate, and mixtures thereof, in water. Suitably, for intrathecal delivery, the osmolarity is within a range compatible with cerebrospinal fluid (e.g., about 275 to about 290); see, e.g., emedicine.medscape.com/article/2093316-overview. Optionally, for intrathecal delivery, a commercially available diluent may be used as a suspending agent, or in combination with another suspending agent and other optional excipients. See, e.g., Elliotts B® solution [Lukare Medical].

In certain embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA

In one embodiment, a frozen composition which contains an rAAV in a buffer solution as described herein, in frozen form, is provided. Optionally, one or more surfactants (e.g., Pluronic F68), stabilizers or preservatives is present in this composition. Suitably, for use, a composition is thawed and titrated to the desired dose with a suitable diluent, e.g., sterile saline or a buffered saline.

In certain embodiments, provided herein is a pharmaceutical composition comprising a vector, such as a rAAV, as described herein and a pharmaceutically acceptable carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. Delivery vehicles such as liposomes, nanocapsules, microparticles, microspheres, lipid particles, vesicles, and the like, may be used for the introduction of the compositions of the present invention into suitable host cells. In particular, the rAAV vector may be formulated for delivery either encapsulated in a lipid particle, a liposome, a vesicle, a nanosphere, or a nanoparticle or the like. In one embodiment, a therapeutically effective amount of said vector is included in the pharmaceutical composition. The selection of the carrier is not a limitation of the present invention. Other conventional pharmaceutically acceptable carrier, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The phrase “pharmaceutically-acceptable” refers to molecular entities and compositions that do not produce an allergic or similar untoward reaction when administered to a host.

As used herein, the term “dosage” or “amount” can refer to the total dosage or amount delivered to the subject in the course of treatment, or the dosage or amount delivered in a single unit (or multiple unit or split dosage) administration.

Also, the replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

In certain embodiments, provided is a pharmaceutical composition comprising a rAAV as described herein in a formulation buffer. In one embodiment, the rAAV is formulated at about 1×10⁹ genome copies (GC)/mL to about 1×10¹⁴ GC/mL. In a further embodiment, the rAAV is formulated at about 3×10⁹ GC/mL to about 3×10¹³ GC/mL. In yet a further embodiment, the rAAV is formulated at about 1×10⁹ GC/mL to about 1×10¹³ GC/mL. In one embodiment, the rAAV is formulated at least about 1×10¹¹ GC/mL. In one embodiment, the pharmaceutical composition comprising a rAAV as described herein is administrable at a dose of about 1×10⁹ GC per gram of brain mass to about 1×10¹⁴ GC per gram of brain mass.

In certain embodiments, the composition may be formulated in a suitable aqueous suspension media (e.g., a buffered saline) for delivery by any suitable route. The compositions provided herein are useful for systemic delivery of high doses of viral vector. For rAAV, a high dose may be at least 1×10¹³ GC or at least 1×10¹⁴ GC. However, for improved safety, the miRNA sequences provided herein may be included in expression cassettes and/or vector genomes which are delivered at other lower doses.

The aqueous suspension or pharmaceutical compositions described herein are designed for delivery to subjects in need thereof by any suitable route or a combination of different routes. In one embodiment, the pharmaceutical composition is formulated for delivery via intracerebroventricular (ICV), intrathecal (IT), or intracisternal injection. In one embodiment, the compositions described herein are designed for delivery to subjects in need thereof by intravenous (IV) injection. Alternatively, other routes of administration may be selected (e.g., oral, inhalation, intranasal, intratracheal, intraarterial, intraocular, intramuscular, and other parenteral routes). In certain embodiments, the composition is delivered by two different routes at essentially the same time.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular, suboccipital/intracisternal, and/or C1-2 puncture. For example, material may be introduced for diffusion throughout the subarachnoid space by means of lumbar puncture. In another example, injection may be into the cisterna magna. Intracisternal delivery may increase vector diffusion and/or reduce toxicity and inflammation caused by the administration. See, e.g., Christian Hinderer et al, Widespread gene transfer in the central nervous system of cynomolgus macaques following delivery of AAV9 into the cisterna magna, Mol Ther Methods Clin Dev. 2014; 1: 14051. Published online 2014 Dec. 10. doi: 10.1038/mtm.2014.51.

As used herein, the terms “intracisternal delivery” or “intracisternal administration” refer to a route of administration for drugs directly into the cerebrospinal fluid of the brain ventricles or within the cisterna magna cerebellomedularis, more specifically via a suboccipital puncture or by direct injection into the cisterna magna or via permanently positioned tube.

It should be understood that the compositions in the pharmaceutical compositions described herein are intended to be applied to other compositions, regiments, aspects, embodiments and methods described across the Specification.

### 6. Methods of Treatment

Provided herein are methods for NPA disease comprising delivering a therapeutically effective amount of a nucleic acid sequence or expression cassette that includes a hSmpd1 coding sequence, as provided herein. In particular, the methods include preventing, treating, and/or ameliorating symptoms of NPA disease by delivering a therapeutically effective amount of a rAAV.hSmpd1 or a composition that includes an hSmpd1 polypeptide described herein to a patient in need thereof. In certain embodiments, a composition comprising an expression cassette as described herein is administrated to a subject in need thereof. In certain embodiments, the expression cassette is delivered via a rAAV.

As used herein, a “therapeutically effective amount” refers to the amount of a composition which delivers an amount of hSmpd1 sufficient to ameliorate or treat one or more of the symptoms of NPA disease. “Treatment” may include preventing the worsening of the symptoms of NPA disease and possibly reversal of one or more of the symptoms thereof. A “therapeutically effective amount” for human patients may be predicted based on an animal model. See, C. Hinderer et al, Molecular Therapy (2014); 22 12, 2018-2027; A. Bradbury, et al, Human Gene Therapy Clinical Development. March 2015, 26(1): 27-37, which are incorporated herein by reference.

In certain embodiments, treatment includes preventing, treating, and/or ameliorating one or more symptoms of NPA.

In certain embodiments, treatment includes replacing or supplementing a patient's defective SMPD1 via rAAV-based gene therapy. As expressed from the rAAV vector described herein, expression levels of at least about 5% of normal levels as detected in the brain, spleen, liver, or other tissue or fluid, may provide therapeutic effect. However, higher expression levels may be achieved. Such expression levels may be from about 5% to about 100% of normal functional human SMPD1 levels. In certain embodiments, higher than normal expression levels may be detected in serum or another biological fluid or tissue.

Suitable volumes for delivery of the compositions provided and concentrations thereof may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

In certain embodiments, the composition comprising a rAAV as described herein is administrable at a dose of about 1×10⁹ GC per gram of brain mass to about 1×10¹⁴ GC per gram of brain mass. In certain embodiments, the rAAV is co-administered systemically at a dose of about 1×10⁹ GC per kg body weight to about 1×10¹³ GC per kg body weight

In certain embodiments, the expression cassette is in a vector genome delivered in an amount of about 1×10⁹ GC per gram of brain mass to about 1×10¹³ genome copies (GC) per gram (g) of brain mass, including all integers or fractional amounts within the range and the endpoints. In another embodiment, the dosage is 1×10¹⁰ GC per gram of brain mass to about 1×10¹³ GC per gram of brain mass. In specific embodiments, the dose of the vector administered to a patient is at least about 1.0×10⁹ GC/g, about 1.5×10⁹ GC/g, about 2.0×10⁹ GC/g, about 2.5×10⁹ GC/g, about 3.0×10⁹ GC/g, about 3.5×10⁹ GC/g, about 4.0×10⁹ GC/g, about 4.5×10⁹ GC/g, about 5.0×10⁹ GC/g, about 5.5×10⁹ GC/g, about 6.0×10⁹ GC/g, about 6.5×10⁹ GC/g, about 7.0×10⁹ GC/g, about 7.5×10⁹ GC/g, about 8.0×10⁹ GC/g, about 8.5×10⁹ GC/g, about 9.0×10⁹ GC/g, about 9.5×10⁹ GC/g, about 1.0×10¹⁰ GC/g, about 1.5×10¹⁰ GC/g, about 2.0×10¹⁰ GC/g, about 2.5×10¹⁰ GC/g, about 3.0×10¹⁰ GC/g, about 3.5×10¹⁰ GC/g, about 4.0×10¹⁰ GC/g, about 4.5×10¹⁰ GC/g, about 5.0×10¹⁰ GC/g, about 5.5×10¹⁰ GC/g, about 6.0×10¹⁰ GC/g, about 6.5×10¹⁰ GC/g, about 7.0×10¹⁰ GC/g, about 7.5×10¹⁰ GC/g, about 8.0×10¹⁰ GC/g, about 8.5×10¹⁰ GC/g, about 9.0×10¹⁰ GC/g, about 9.5×10¹⁰ GC/g, about 1.0×10¹¹ GC/g, about 1.5×10¹¹ GC/g, about 2.0×10¹¹ GC/g, about 2.5×10¹¹ GC/g, about 3.0×10¹¹ GC/g, about 3.5×10¹¹ GC/g, about 4.0×10¹¹ GC/g, about 4.5×10¹¹ GC/g, about 5.0×10¹¹ GC/g, about 5.5×10¹¹ GC/g, about 6.0×10¹¹ GC/g, about 6.5×10¹¹ GC/g, about 7.0×10¹¹ GC/g, about 7.5×10¹¹ GC/g, about 8.0×10¹¹ GC/g, about 8.5×10¹¹ GC/g, about 9.0×10¹¹ GC/g, about 9.5×10¹¹ GC/g, about 1.0×10¹² GC/g, about 1.5×10¹² GC/g, about 2.0×10¹² GC/g, about 2.5×10¹² GC/g, about 3.0×10¹² GC/g, about 3.5×10¹² GC/g, about 4.0×10¹² GC/g, about 4.5×10¹² GC/g, about 5.0×10¹² GC/g, about 5.5×10¹² GC/g, about 6.0×10¹² GC/g, about 6.5×10¹² GC/g, about 7.0×10¹² GC/g, about 7.5×10¹² GC/g, about 8.0×10¹² GC/g, about 8.5×10¹² GC/g, about 9.0×10¹² GC/g, about 9.5×10¹² GC/g, about 1.0×10¹³ GC/g, about 1.5×10¹³ GC/g, about 2.0×10¹³ GC/g, about 2.5×10¹³ GC/g, about 3.0×10¹³ GC/g, about 3.5×10¹³ GC/g, about 4.0×10¹³ GC/g, about 4.5×10¹³ GC/g, about 5.0×10¹³ GC/g, about 5.5×10¹³ GC/g, about 6.0×10¹³ GC/g, about 6.5×10¹³ GC/g, about 7.0×10¹³ GC/g, about 7.5×10¹³ GC/g, about 8.0×10¹³ GC/g, about 8.5×10¹³ GC/g, about 9.0×10¹³ GC/g, about 9.5×10¹³ GC/g, or about 1.0×10¹⁴ GC/g brain mass.

In certain embodiments, the compositions provided herein are administered in combination an immunosuppressant. Currently, immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin.

The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In certain embodiments, the immunosuppressive therapy may be started 0, 1, 2, 7, or more days prior to the gene therapy administration. Such therapy may involve co-administration of two or more drugs, the (e.g., prednisone, mycophenolate mofetil (MMF) and/or sirolimus (i.e., rapamycin)) on the same day. One or more of these drugs may be continued after gene therapy administration, at the same dose or an adjusted dose.

In certain embodiments, a rAAV as provided herein is administered in combination with a therapy (co-therapy), such as an enzyme-replacement therapy (e.g., SMPD1 (also known as ASMase) enzyme replacement), chaperone therapy, substrate reduction therapy, and/or in combination with antihistamines or other medications which reduce the chance of infusion related reactions. In certain embodiments, the co-therapy is a functional hSmpd1 protein. Administration may be oral or by intravenous infusion to an outpatient and may be include dosages suitable for daily, every other day, weekly, every two weeks (e.g., 0.2 mg/kg body weight), monthly, or bimonthly administration. Appropriate therapeutically effective dosages of the co-therapies are selected by the treating clinician and include from about 1 g/kg to about 500 mg/kg, from about 10 mg/kg to about 100 mg/kg, from about 20 mg/kg to about 100 mg/kg and approximately 20 mg/kg to approximately 50 mg/kg. In some embodiments, a suitable therapeutic dose is selected from, for example, 0.5, 0.75, 1, 5, 10, 15, 20, 30, 40, 50, 60, 70, 100, 150, 200, 250, 300, 400, or 500 mg/kg.

In certain embodiments, newborn babies (3 months old or younger) are treated in accordance with the methods described herein. In certain embodiments, babies that are 3 months old to 9 months old are treated in accordance with the methods described herein. In certain embodiments, children that are 9 months old to 36 months old are treated in accordance with the methods described herein. In certain embodiments, children that are 3 years old to 12 years old are treated in accordance with the methods described herein. In certain embodiments, children that are 12 years old to 18 years old are treated in accordance with the methods described herein. In certain embodiments, adults that are 18 years old or older are treated in accordance with the methods described herein.

In one embodiment, a patient with NPA disease is a male or female of at least about 3 months to less than 12 months of age. In another embodiment, the patient with NPA disease is a male or female and at least about 6 years to up to 18 years of age. In other embodiments, the subjects may be older or younger, and may be male or female.

In certain embodiments, provided herein is a use of an rAAV, as described herein, in preparing a medicament for treatment of Niemann Pick A and/or improving the symptoms thereof by mitigating weight loss or cachexia, mitigating loss of motor function, mitigating loss of cognitive function and prolonging survival.

### 7. Device and Kit

In one aspect, the vectors provided herein may be administered intrathecally via the method and/or the device described, e.g., in WO 2017/136500, which is incorporated herein by reference in its entirety. Alternatively, other devices and methods may be selected. In summary, the method comprises the steps of advancing a spinal needle into the cisterna magna of a patient, connecting a length of flexible tubing to a proximal hub of the spinal needle and an output port of a valve to a proximal end of the flexible tubing, and after said advancing and connecting steps and after permitting the tubing to be self-primed with the patient's cerebrospinal fluid, connecting a first vessel containing an amount of isotonic solution to a flush inlet port of the valve and thereafter connecting a second vessel containing an amount of a pharmaceutical composition to a vector inlet port of the valve. After connecting the first and second vessels to the valve, a path for fluid flow is opened between the vector inlet port and the outlet port of the valve and the pharmaceutical composition is injected into the patient through the spinal needle, and after injecting the pharmaceutical composition, a path for fluid flow is opened through the flush inlet port and the outlet port of the valve and the isotonic solution is injected into the spinal needle to flush the pharmaceutical composition into the patient. This method and this device may each optionally be used for intrathecal delivery of the compositions provided herein. Alternatively, other methods and devices may be used for such intrathecal delivery.

In certain embodiments, a kit is provided which includes a concentrated vector suspended in a formulation (optionally frozen), optional dilution buffer, and devices and components required for intravenous, intrathecal, intracerebroventricular, or intracisternal administration. In one embodiment, the kit provides sufficient buffer to allow for injection. Such buffer may allow for about a 1:1 to a 1:5 dilution of the concentrated vector, or more. Such a kit may include additional non-vector based active components where a combination therapy is utilized and/or anti-histamines, immunomodulators, or the like. In other embodiments, higher or lower amounts of buffer or sterile water are included to allow for dose titration and other adjustments by the treating clinician. In still other embodiments, one or more components of the device are included in the kit. Suitable dilution buffer is available, such as, a saline, a phosphate buffered saline (PBS) or a glycerol/PBS.

It should be understood that the compositions in kits described herein are intended to be applied to other compositions, regiments, aspects, embodiments and methods described across the Specification.

As used herein, the term “biological sample” refers to any cell, biological fluid, or tissue. Suitable samples for use in this invention may include, without limitation, whole blood, leukocytes, fibroblasts, serum, urine, plasma, saliva, bone marrow, cerebrospinal fluid, amniotic fluid, and skin cells. Such samples may further be diluted with saline, buffer or a physiologically acceptable diluent. Alternatively, such samples are concentrated by conventional means.

With regard to the description of these inventions, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

“Patient” or “subject” as used herein refers to a male or female human and animal models used for clinical research. In certain embodiments, the subject of these methods and compositions is a human diagnosed with NPA disease. In further embodiments, the human subject of these methods and compositions is a prenatal, a newborn, an infant, a toddler, a preschool-aged child, a grade-school-aged child, a teen, a young adult, or an adult.

“Comprising” is a term meaning inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention. It should be understood that while various embodiments in the specification are presented using “comprising” language, under various circumstances, a related embodiment is also described using “consisting of” or “consisting essentially of” language.

A reference to “one embodiment”, “another embodiment”, or “a certain embodiment” in describing an embodiment does not imply that the referenced embodiment is mutually exclusive with another embodiment (e.g., an embodiment described before the referenced embodiment), unless expressly specified otherwise.

It is to be noted that the term “a” or “an”, refers to one or more, for example, “an expression cassette”, is understood to represent one or more expression cassette (s). As such, the terms “a” (or “an”), “one or more,” and “at least one” are used interchangeably herein.

As used herein, the term “about” means a variability of plus or minus 10% from the reference given, unless otherwise specified.

It should be understood that the compositions in the devices described herein are intended to be applied to other compositions, regiments, aspects, embodiments and methods described across the Specification.

### Examples

The invention is now described with reference to the following examples. These examples are provided for the purpose of illustration only and the invention should in no way be construed as being limited to these examples but rather should be construed to encompass any and all variations that become evident as a result of the teachings provided herein.

### Example 1: A rAAV.hSMPD1 for Treatment of NPC

Cis plasmid containing the vector genome is generated by cloning the hSMPD1V36A coding sequence of SEQ ID NO: 4 or the hSMPD1 having the V at position 36 of SEQ ID NO: 5. The vector genome was designed to have a shortened AAV2 (130 bp) 5′ ITR at the extreme 5′ end of the vector genome, the expression cassette, and a shorted AAV2 (130 bp) 3′ ITR at the extreme 3′ end of the vector genome. An expression cassette containing the CB7 promoter, a chimeric intron, the coding sequence, and a rabbit beta globin polyA (CB7.CI.hSMPD1co.RBG, SEQ ID NO: 8 or SEQ ID NO: 9) is used to generate rAAV having a vector genome of SEQ ID NO: 11 or SEQ ID NO: 11. An expression cassette containing the UbC promoter, the coding sequence, and a SV40 polyA sequence (UbC.hSMPD1co.SV40, SEQ ID NO: 14) is used to generate rAAV having a vector genome of SEQ ID NO: 15. Recombinant AAV9 viral particles are generated by triple transfection of a packaging 293 host cell with one of the above cis plasmids, a trans plasmid containing the AA2 rep functions and the AAV capsid coding sequence, and a helper plasmid.

The resulting rAAV.hSMPD1 are used in the comparative study of Example 2.

### Example 2: Delivery of AAV.hSMPD1 Via Intracerebroventricular (ICV) Injection for the Treatment of NPA

A Smpd1 KO mouse model was generated by CRISPR/Cas9 through deletion of exon 2 of the mouse Smpd1 gene (Jackson Labs). The resulting animals are a good model of NPA disease. Similar to published models, mice are lethargic, have gait abnormalities, CNS/respiratory/cardiac involvement, and untreated animals show significant storage material in target tissues.

1 month old Smpd1 KO mice received 6×10¹⁰ genomic copies of AAV. CB7.hSMPD1 or AAV.Ubc.hSMPD1 via ICV injection. Companion control mice received PBS. Mice were observed daily and weighed weekly. To assess motor coordination, mice were given rotarod performance tests monthly. Smpd1 KO mice that received PBS displayed a decrease in latency to fall over time. This behavioral defect was rescued by the delivery of AAV. CB7.hSMPD1 or AAV.Ubc.hSMPD1 into Smpd1 KO mice. Mice were euthanized 5.3 months post-injection. Brain, liver, spleen and lung tissues were harvested, fixed and processed for histological analyses. Immunostaining on brain sections with filipin to detect cholesterol deposits revealed an abundant amount of cholesterol in Smpd1 KO mice. The amount of stored cholesterol was greatly reduced in Smpd1 KO mice that received AAV. CB7.hSMPD1 or AAV.Ubc.hSMPD1. To detect the presence of lipid-filled macrophages, oil red staining was carried out on liver, spleen and lung tissues. The presence of lipid-filled macrophages was seen in all three tissues derived from Smpd1 KO mice. A decrease or absence of the lipid-filled macrophages was evident in Smpd1 KO mice that received AAV.CB7.hSMPD1 or AAV.Ubc.hSMPD1. Other measures of efficacy may include sphingomyelin quantification (liver/brain/spleen) and a chitinase assay (biomarker of disease severity).

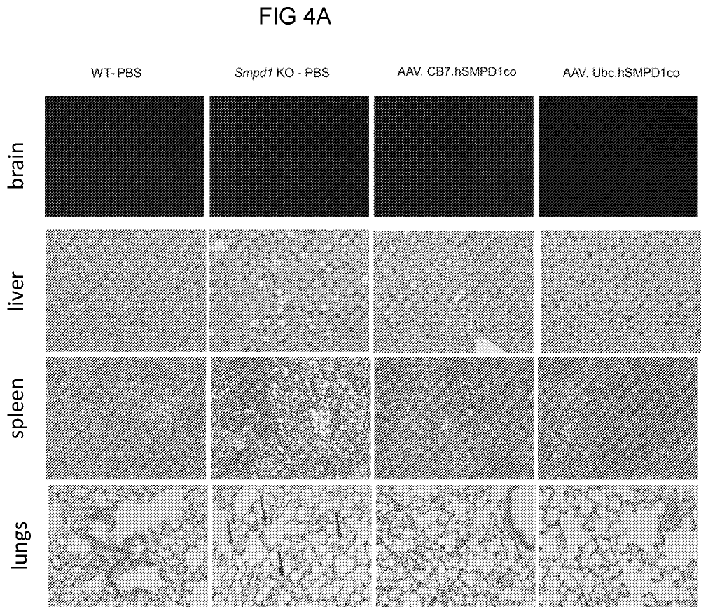

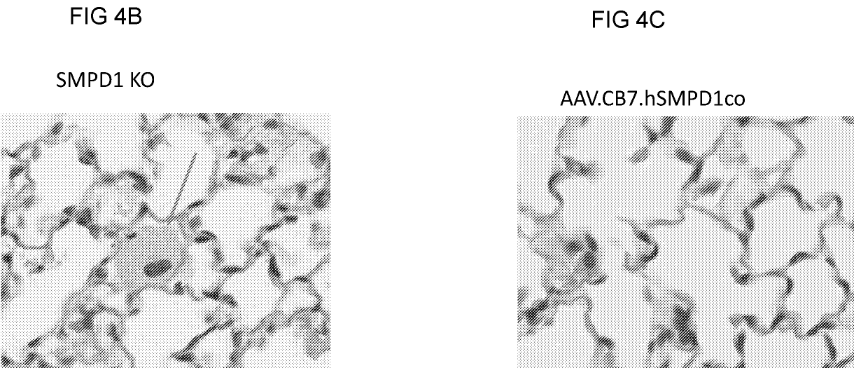

These results are provided in FIGS. 4A-4C.

### Example 3: Delivery of AAV.CB76.hSMPD1 with and without DRG De-Targeting Sequences in Non-Human Primates

A further engineered sequence, SEQ ID NO: 22 was generated (encoding SEQ ID NO: 2 and 23) and selected for use in generating further recombinant AAV vectors. rAAVhu68.CB7.hSMPD1coV2 vectors were prepared with or without 4×miR182 targets (binding sequences). A cis plasmid was designed to contain a vector genome comprising an AAV2-5′ ITR, an expression comprising a hSMPD1coV2 coding sequence (SEQ ID NO: 22), with or without 4 copies (4×) miR182 targets (binding sites) (SEQ ID NO: 24), and an AAV2-5′ ITR. The cis-plasmid is co-transfected into HEK293 packaging host cells with a trans plasmid encoding rep and the encoding the AAVhu68 VP1 capsid protein under the control of sequences which direct expression thereof in the 293 cell, and a cis plasmid comprising adenovirus helper sequences for replication and packaging of the vector genome into the hu68 capsid using conventional triple transfection production methods. The resulting viral particles are termed herein, AAVhu68.CB7.hSMPD1co (without miR binding sites) or AAV.CB7.hSMPD1co.miR-TS (with 4×miR182 biding sites). The expression cassette for the CB7.CI.hSMPDcoV2.RBG is provided in SEQ ID NO: 21, and contains a CB7 promoter element (SEQ ID NO: 26), the hCMPD1coV2 coding sequence (SEQ ID NO: 22) and a rabbit globin poly A (SEQ ID NO: 7). The vector genome for the ITR.CB7.CI.hSMPDcoV2.RGB.ITR is provided in SEQ ID NO: 20. The expression cassette for the CB7.CI.hSMPDcoV2.miR-TS is provided in SEQ ID NO: 18, and contains a CB7 promoter element (SEQ ID NO: 26), the hCMPD1coV2 coding sequence (SEQ ID NO: 22), 4×miR182 binding sites (SEQ ID NO: 28) and a rabbit globin poly A (SEQ ID NO: 7). The vector genome for the ITR.CB7.CI.hSMPDcoV2.miR-TS comprises SEQ ID NO: 18.

Rhesus macaques were injected ICM with 3×10¹³ GC AAVhu68.CB7.hSMPD1co or AAV.CB7.hSMPD1co.miR-TS. The animals are observed daily and monitored for DRG pathology via nerve conduction studies: SNAP amplitude and by detection of a biomarker. Two-months post-injection, the animals are necropsied and assessed for biodistribution, ASMD expression, and DRG pathology.

### Sequence Listing Free Text

The following information is provided for sequences containing free text under numeric identifier <223>.

| SEQ ID NO: | Free Text under <223> |
|---|---|
| 3 | <223> hSMB1.V36-R506G |
| 4 | <223> Engineered hSMPD1.V36 coding sequence |
| 5 | <223> hSMPD1.V36 |
| 6 | <223> CB promoter |
| 7 | <223> Rabbit globin polyA |
| 8 | <223> CB7.CI.hSMPD1co(TY).RBG |
| | <220> |
| | <221> misc_feature |
| | <222> (1)..(382) |
| | <223> CMV IE enhancer |
| | <220> |
| | <221> misc_feature |
| | <222> (1)..(666) |
| | <223> CB7 hybrid promoter |
| | <220> |
| | <221> misc_feature |
| | <222> (385)..(666) |
| | <223> CB promoter |
| | <220> |
| | <221> misc_feature |
| | <222> (759)..(1731) |
| | <223> chicken beta actin intron |
| | <220> |
| | <221> misc_feature |
| | <222> (1749)..(3641) |
| | <223> hSMPD1.V36A.co |
| | <220> |
| | <221> misc_feature |
| | <222> (3675)..(3801) |
| | <223> Rabbit beta globin polyA |
| 9 | <223> ITR.CB7.CI.hSMPD1co(TY).RBG.ITR |
| | <220> |
| | <221> misc_feature |
| | <222> (1)..(130) |
| | <223> 5′ ITR |
| | <220> |
| | <221> misc_feature |
| | <222> (198)..(579) |
| | <223> CMV IE promoter |
| | <220> |
| | <221> misc_feature |
| | <222> (198)..(863) |
| | <223> CB7 hybrid promoter |
| | <220> |
| | <221> misc_feature |
| | <222> (582)..(863) |
| | <223> CB promoter |
| | <220> |
| | <221> misc_feature |
| | <222> (956)..(1928) |
| | <223> chicken beta-actin intron |
| | <220> |
| | <221> misc_feature |
| | <222> (1946)..(3838) |
| | <223> hSMPD1.V36A.co |
| | <220> |
| | <221> misc_feature |
| | <222> (3872)..(3998) |
| | <223> rabbit gobin polyA |
| | <220> |
| | <221> misc_feature |
| | <222> (4087)..(4216) |
| | <223> 3′ ITR |
| 10 | <223> CB7.CI.hSMPD1.V36co.RBG |
| | <220> |
| | <221> misc_feature |
| | <222> (1)..(382) |
| | <223> CMV IE enhancer |
| | <220> |
| | <221> misc_feature |
| | <222> (1)..(666) |
| | <223> CB7 hybrid promoter |
| | <220> |
| | <221> misc_feature |
| | <222> (385)..(666) |
| | <223> CB promoter |
| | <220> |
| | <221> misc_feature |
| | <222> (759)..(1731) |
| | <223> chicken beta actin inron |
| | <220> |
| | <221> misc_feature |
| | <222> (1749)..(3641) |
| | <223> hSMPD1.V36.co |
| | <220> |
| | <221> misc_feature |
| | <222> (3675)..(3801) |
| | <223> rabbit beta globin polyA |
| 11 | <223> ITR.CB7.CI.hSMPD1coV36.RBG.ITR |
| | <220> |
| | <221> misc_feature |
| | <222> (1)..(130) |
| | <223> 5′ ITR |
| | <220> |
| | <221> misc_feature |
| | <222> (198)..(863) |
| | <223> CB7 hybrid promoter |
| | <220> |
| | <221> misc_feature |
| | <222> (198)..(579) |
| | <223> CMV IE promoter |
| | <220> |
| | <221> misc_feature |
| | <222> (583)..(863) |
| | <223> CB promoter |
| | <220> |
| | <221> misc_feature |
| | <222> (956)..(1928) |
| | <223> chicken beta-actin intron |
| | <220> |
| | <221> misc_feature |
| | <222> (1946)..(3838) |
| | <223> hSMPD1,V36co |
| | <220> |
| | <221> misc_feature |
| | <222> (3872)..(3998) |
| | <223> rabbit globin poly A |
| | <220> |
| | <221> misc_feature |
| | <222> (4088)..(4216) |
| | <223> 3′ ITR |
| 12 | <223> UbC Promoter |
| 13 | <223> SV40 late polyA signal |
| 14 | <223> UbC.hSMPD1co.SV40 |
| | <220> |
| | <221> misc_feature |
| | <222> (1)..(1229) |
| | <223> UbC promoter |
| | <220> |
| | <221> misc_feature |
| | <222> (1323)..(1455) |
| | <223> Promega Chimeric Intron |
| | <220> |
| | <221> misc_feature |
| | <222> (1540)..(3432) |
| | <223> hSMPD1.V36A.co |
| | <220> |
| | <221> misc_feature |
| | <222> (3464)..(3695) |
| | <223> SV40 late polyA signal |
| 15 | <223> ITR.UbC.hSMPD1co.SV40.ITR |
| | <220> |
| | <221> misc_feature |
| | <222> (1)..(130) |
| | <223> 5′ ITR |
| | <220> |
| | <221> misc_feature |
| | <222> (191)..(1419) |
| | <223> UbC promoter |
| | <220> |
| | <221> misc_feature |
| | <222> (1513)..(1645) |
| | <223> Promega chimeric intron |
| | <220> |
| | <221> misc_feature |
| | <222> (1730)..(3622) |
| | <223> hSMPD1.V36Aco |
| | <220> |
| | <221> misc_feature |
| | <222> (3654)..(3885) |
| | <223> SV40 late polyA signal |
| | <220> |
| | <221> misc_feature |
| | <222> (3950)..(4079) |
| | <223> 3 ITR |
| 16 | <223> AAVhu68 vp1 capsid of Homo Sapiens origin |
| 17 | <223> Synthetic Construct |
| 18 | <223> ITR.CB7.CI.hSMPD1coV2.4xmiR182.rBG.ITR |
| | <220> |
| | <221> misc_feature |
| | <222> (1)..(130) |
| | <223> 5′ ITR |
| | <220> |
| | <221> misc_feature |
| | <222> (198)..(579) |
| | <223> CMV IE enhancer |
| | <220> |
| | <221> misc_feature |
| | <222> (198)..(863) |
| | <223> CB7 hybrid promoter |
| | <220> |
| | <221> misc_feature |
| | <222> (582)..(863) |
| | <223> CB promoter |
| | <220> |
| | <221> misc_feature |
| | <222> (956)..(1928) |
| | <223> chicken beta actin intron |
| | <220> |
| | <221> misc_feature |
| | <222> (1934)..(3843) |
| | <223> hSMPD1coV2 |
| | <220> |
| | <221> misc_feature |
| | <222> (3845)..(3868) |
| | <223> miR182 |
| | <220> |
| | <221> misc_feature |
| | <222> (3873)..(3896) |
| | <223> miR182 |
| | <220> |
| | <221> misc_feature |
| | <222> (3903)..(3926) |
| | <223> miR182 |
| | <220> |
| | <221> misc_feature |
| | <222> (3933)..(3956) |
| | <223> miR182 |
| | <220> |
| | <221> misc_feature |
| | <222> (3968)..(4094) |
| | <223> Rabbit beta globin polyA |
| | <220> |
| | <221> misc_feature |
| | <222> (4183)..(4312) |
| | <223> 3′ ITR |
| 19 | <223> CB7.CI.hSMPD1coV2.4xmiR182.rBG |
| | <220> |
| | <221> misc_feature |
| | <222> (1)..(382) |
| | <223> CMV IE enhancer |
| | <220> |
| | <221> misc_feature |
| | <222> (1)..(666) |
| | <223> CB7 hybrid promoter |
| | <220> |
| | <221> misc_feature |
| | <222> (385)..(666) |
| | <223> CB promoter |
| | <220> |
| | <221> misc_feature |
| | <222> (759)..(1731) |
| | <223> chicken beta actin intron |
| | <220> |
| | <221> misc_feature |
| | <222> (1743)..(1748) |
| | <223> Kozak |
| | <220> |
| | <221> misc_feature |
| | <222> (1749)..(3641) |
| | <223> hSMPD1coV2 |
| | <220> |
| | <221> misc_feature |
| | <222> (3648)..(3671) |
| | <223> miR182 |
| | <220> |
| | <221> misc_feature |
| | <222> (3676)..(3699) |
| | <223> miR182 |
| | <220> |
| | <221> misc_feature |
| | <222> (3706)..(3729) |
| | <223> miR182 |
| | <220> |
| | <221> misc_feature |
| | <222> (3736)..(3759) |
| | <223> miR182 |
| | <220> |
| | <221> misc_feature |
| | <222> (3771)..(3897) |
| | <223> rabbit globin polyA |
| 20 | <223> ITR.CB7.CI.hSMPD1cov2.RBG.ITR |
| | <220> |
| | <221> misc_feature |
| | <222> (1)..(130) |
| | <223> 5′ITR |
| | <220> |
| | <221> misc_feature |
| | <222> (198)..(579) |
| | <223> CMV IE enhancer |
| | <220> |
| | <221> misc_feature |
| | <222> (198)..(863) |
| | <223> CB7 hybrid promoter |
| | <220> |
| | <221> misc_feature |
| | <222> (582)..(863) |
| | <223> CB promoter |
| | <220> |
| | <221> misc_feature |
| | <222> (956)..(1928) |
| | <223> chicken beta actin intron |
| | <220> |
| | <221> misc_feature |
| | <222> (1940)..(19456) |
| | <223> Kozak |
| | <220> |
| | <221> misc_feature |
| | <222> (1946)..(3838) |
| | <223> hSMPD1coV2 |
| | <220> |
| | <221> misc_feature |
| | <222> (3899)..(4025) |
| | <223> rabbit beta globin polyA |
| | <220> |
| | <221> misc_feature |
| | <222> (4114)..(4243) |
| | <223> 3′ ITR |
| 21 | <223> CB7.CI.hSMPD1cov2.RBG |
| | <220> |
| | <221> misc_feature |
| | <222> (1)..(382) |
| | <223> CMV IE enhancer |
| | <220> |
| | <221> misc_feature |
| | <222> (1)..(666) |
| | <223> CB7 hybrid promoter |
| | <220> |
| | <221> misc_feature |
| | <222> (385)..(666) |
| | <223> CB promoter |
| | <220> |
| | <221> misc_feature |
| | <222> (759)..(1731) |
| | <223> chicken beta-actin intron |
| | <220> |
| | <221> misc_feature |
| | <222> (1743)..(1748) |
| | <223> Kozak |
| | <220> |
| | <221> misc_feature |
| | <222> (1749)..(3641) |
| | <223> hSMPD1coV2 |
| | <220> |
| | <221> misc_feature |
| | <222> (3702)..(3828) |
| | <223> Rabbit beta globin polyA |
| 22 | <223> hSMPD1coV2 |
| | <220> |
| | <221> CDS |
| | <222> (1)..(1893) |
| | <223> hSMPD1coV2 |
| 23 | <223> Synthetic Construct |
| 24 | <223> miR182 |
| 25 | <223> cytomegalovirus (CMV) IE enhancer |
| 26 | <223> CB7 hybrid promoter |
| 27 | <223> chicken beta-actin intron |
| 28 | <223> 4 tandem repeats of miR182 with linker sequences |
| | <220> |
| | <221> misc_feature |
| | <222> (1)..(24) |
| | <223> miR182 |
| | <220> |
| | <221> misc_feature |
| | <222> (29)..(52) |
| | <223> miR182 |
| | <220> |
| | <221> misc_feature |
| | <222> (59)..(82) |
| | <223> miR182 |
| | <220> |
| | <221> misc_feature |
| | <222> (89)..(112) |
| | <223> miR182 |
| 30 | <223> native signal peptide of hSMPD1 (aa 1 to 46) |
| 31 | <223> hSMPD1 mature protein (aa 47 to about 629) |

All patent and non-patent publications cited in this specification are incorporated herein by reference in their entireties, as is U.S. Provisional Patent Application No. 63/144,103, filed Feb. 1, 2021, U.S. Provisional Patent Application No. 63/286,939, filed Dec. 7, 2022, and the SEQ ID NOs that are referenced herein and which appear in the appended Sequence Listing, 21-9627PCT_NPA_ST25, generated Jan. 21, 2022 (78 kb) are incorporated by reference. While the invention has been described with reference to particular embodiments, it will be appreciated that modifications can be made without departing from the spirit of the invention. Such modifications are intended to fall within the scope of the appended claims.

1. A recombinant AAV (rAAV) comprising an AAV capsid and a vector genome packaged therein, wherein the vector genome comprises an engineered nucleic acid sequence encoding a human acid sphingomyelinase (hSMPD1) operably linked to a regulatory sequence which directs expression of the hSMPD1, and an AAV 3′ ITR, wherein the hSMPD1 coding sequence is SEQ ID NO: 22 or a coding sequence at least 99% identical to SEQ ID NO: 22 which encodes functional hSMPD1 protein, SEQ ID NO: 5 or a coding sequence at least 90% identical to SEQ ID NO: 5 which encodes functional hSMPD1 protein, or SEQ ID NO: 4 or a coding sequence at least 90% identical to SEQ ID NO: 4 which encodes a functional hSMPD1 protein.

2. The rAAV according to claim 1, wherein the hSMPD1 coding sequence encodes the hSMD1 protein having an Ala or a Val at position 36 with reference to the number of SEQ ID NO: 2.

3. The rAAV according to claim 1, wherein the hSMPD1 protein has the sequence of SEQ ID NO: 2, SEQ ID NO: 23, SEQ ID NO: 3, or SEQ ID NO: 1.

4. The rAAV according to claim 1, wherein the hSMPD1 protein comprises an exogenous leader sequence fused to an hSMPD1 protein comprising amino acids 47 to 631 of SEQ ID NO: 2 (or SEQ ID NO: 31), SEQ ID NO: 23, SEQ ID NO: 3, or SEQ ID NO: 1.

5. The rAAV according to claim 1, wherein the regulatory sequences comprise a UbC or a CB7 promoter.

6. The rAAV according to claim 1, wherein the regulatory sequences comprise a SV40 late or a rabbit beta globin polyadenylation site.

7. The rAAV according to claim 1, wherein the AAV vector genome comprises an expression cassette comprising a CB7 hybrid promoter, an intron, the hSMPD1 coding sequence, and a rabbit beta globin sequence, and, optionally, four or more miR182 or miR183 binding sites.

8. The rAAV according to claim 1, wherein the vector genome comprises the sequence of SEQ ID NO: 21, SEQ ID NO: 19, SEQ ID NO: 10, or SEQ ID NO: 8.

9. The rAAV according to claim 1, wherein the vector genome comprises the sequence of SEQ ID NO: 20, SEQ ID NO: 18, SEQ ID NO: 9, or SEQ ID NO: 11.

10. The rAAV according to claim 1, wherein the AAV vector genome comprises a UbC promoter, the hSMPD1 coding sequence, and a SV40 late polyadenylation sequence.

11. The rAAV according to claim 10, wherein the vector genome comprises the sequence of SEQ ID NO: 14 or 15.

12. The rAAV according to claim 1, wherein the AAV vector genome further comprises full-length AAV2 inverted terminal repeat sequences.

13. The rAAV according to claim 1, wherein the AAV capsid is an AAVhu68 capsid.

14. A pharmaceutical composition comprising a population of rAAV according to claim 1 in a formulation buffer.

15. The pharmaceutical composition according to claim 14, which comprises the rAAV encoding the hSMPD1 having an Ala in position 36, the rAAV encoding hSMPD1 having a Val in position 36, or a combination thereof.

16. The pharmaceutical composition according to claim 14, which is suitable for co-administering with a functional hSMPD1 protein.

17. The pharmaceutical composition according to claim 14, which is formulated for delivery via intracerebroventricular (ICV), intrathecal (IT), intracisternal or intravenous (IV) injection.

18-20. (canceled)

21. A nucleic acid molecule comprising an engineered nucleic acid sequence encoding a human acid sphingomyelinase (hSMPD1) operably linked to sequences which regulate expression of the hSMPD1, wherein the hSMPD1 coding sequence is SEQ ID NO: 22 or a coding sequence at least 90% identical to SEQ ID NO: 22 which encodes functional hSMPD1; SEQ ID NO: 4 or a coding sequence at least 90% identical to SEQ ID NO: 4 which encodes functional hSMPD1, or SEQ ID NO: 5 or a coding sequence at least 90% identical to SEQ ID NO: 5 which encodes a functional hSMPD1 protein.

22. A viral or non-viral vector comprising the nucleic acid molecule according to claim 21.

23. A plasmid comprising the nucleic acid molecule according to claim 21.

24. The plasmid according to claim 23 which comprises a vector genome for packaging into an rAAV, where the vector gene comprises the engineered nucleic acid sequence, regulatory sequences, and comprises a 5′ inverted terminal repeat sequence (ITR) and a 3′ ITR, at the extreme 5′ end and the extreme 3′ end, respectively, of the vector genome.

25. A packaging host cell comprising the plasmid according to claim 22, AAV rep coding sequences and AAV cap coding sequences operably linked to regulatory sequences which direct their expression, and helper functions to enable replication and packaging of the vector genome into the AAV capsid.

26. A method of treating a subject diagnosed with Neimann Pick A and/or improving the symptoms thereof by mitigating weight loss or cachexia, mitigating loss of motor function, mitigating loss of cognitive function and prolonging survival in a subject in need thereof, comprising administering to the subject a pharmaceutical composition comprising a population of rAAV according to claim 1 in a formulation buffer.
